(19) United States
(12) Patent Application Publication
(10) Pub. No.: US 2020/0308550 A1
(43) Pub. Date: Oct. 1, 2020

(54) TUMOR ORGANOID MODEL

(71) Applicant: IMBA - INSTITUT FÜR MOLEKULARE BIOTECHNOLOGIE GMBH, Vienna (AT)

(72) Inventors: Jürgen KNOBLICH, Vienna (AT); Shan BIAN, Vienna (AT)

(21) Appl. No.: 16/646,548

(22) PCT Filed: Sep. 11, 2018
(86) PCT No.: PCT/EP2018/074382

§ 371 Date: Mar. 11, 2020

(30) Foreign Application Priority Data
Sep. 11, 2017 (EP) 17190447.7

(51) Int. Cl. C12N 5/09; C12N 5/079; C12N 5/071; G01N 33/50

(52) U.S. Cl. CPC C12N 5/0693; C12N 2533/90; C12N 5/0697; G01N 33/5011; G01N 33/5058; G01N 33/5088; C12N 2501/115; C12N 2501/385; C12N 2501/606; C12N 2503/02; C12N 2506/02; C12N 2506/45; C12N 2510/04; C12N 2513/00; C12N 5/0618

(57) ABSTRACT

A method of generating an artificial 3D tissue culture of a cancer grown in non-cancerous tissue, includes the steps of providing an aggregate of pluripotent stem cells or progenitor cells, culturing and expanding the cells in a 3D biocompatible matrix, wherein the cells are allowed to differentiate to develop the aggregate into a tissue culture of a desired size; wherein at least a portion of the cells are subjected to cancerogenesis by expressing a oncogene and/or by suppressing a tumor suppressor gene during any of the steps or in the tissue culture, and further including the step of allowing the cells with an expressed oncogene or suppressed tumor suppressor to develop into cancerous cells; drug screening methods; oncolytic virus screening methods; a 3D tissue culture; and a kit for performing the inventive methods.

The invention relates to the field of artificial tissue models grown in vitro.

### BACKGROUND OF THE INVENTION

Malignant brain tumors are among the most devastating cancers with almost negligible survival rates that have not improved in decades. Development of suitable brain cancer models and effective therapies is challenging due to their enormous genetic (McLendon et al., 2008, Nature, 455, 1061-8) and morphological (Louis et al., 2016, Acta Neuropathol, 131, 803-20) heterogeneity. In addition, the obvious morphological and physiological differences between human and rodent brains limit the development of appropriate animal models (Lui et al., 2011, Cell, 146, 18-36). Human brain cancer cell lines as well as cancer stem cells cultured in 2D (Hu et al., 2016, Cell, 167, 1281-1295.e18) have served as surrogate models but do not recapitulate the 3D tumor environment.

The recent development of organoid culture models has opened new avenues for modelling disease directly in human tissues. Recapitulating either organ regeneration from adult stem cells (ASCs) (Sato et al., 2009, Nature, 459, 262-5) or organ development from pluripotent stem cells (PSCs) (Kelava and Lancaster, 2016, Cell Stem Cell, 18, 736-48), organoids resemble organ histology and physiology in a strikingly accurate manner (Lancaster and Knoblich, 2014, Science, 345, 1247125).

US 2014/302491 A1 relates to a culture system for long term cultures of mammalian tissues.

Xiaolei et al., Cell Stem Cell 18 (1) (2016): 25-38 is a review on to stem-cell based organoids.

WO 00/75286 A2 describes in vitro 3D models of various cancer tissues, which can be used for screening applications.

Ridder et al., International Journal of Cancer Research and Treatment 17 (6B) (1997), relates to brain tumor spheroids that are attached to human dermal spheroids in order to test tumor invasiveness.

Nygaard et al., Journal of Neurosurgery 89 (3) (1998): 2843-2857, describes spheroids of glioblastoma that are cocultured with rat brain aggregates.

Zhu et al., The Journal of Experimental Medicine 214 (10) (2017): 2843-2857, describes an oncolytic effect of Zika virus against glioblastoma.

Wang et al., PLOS ONE 9 (4) (2014): 1, describes studying 3D organoids as disease development models.

Organoids have been used to model various human diseases (Johnson and Hockemeyer, 2015, Curr Opin Cell Biol, 37, 84-90), including cancer (Neal and Kuo, 2016, Annu Rev Pathol). For ASC-derived neoplastic organoids, this can be achieved by using genetically modified ASCs (Barker et al., 2009, Nature, 457, 608-11; Drost et al., 2015, Nature, 521, 43-7; Matano et al., 2015, Nature Medicine, 21, 256-62) or primary tumors (Boj et al., 2015, Cell, 160, 324-38) as a starting material. For PSC-derived organoids, however, this approach is difficult as the growth requirements of these organoids are often not compatible with adult tumor cells or will impose selective pressure on them.

Therefore, there remains a goal to produce improved cancer model cultures, especially 3D cultures of cancer and in particular to model additional characteristics of cancer in vitro cultures that closely resemble in vivo cancer.

### SUMMARY OF THE INVENTION

In particular, the invention has the goal of recapitulation of life-like circumstances during cancer development. This goal is solved by introducing tumorigenesis together with development of normal, non-cancerous tissues in organoids.

The invention provides a method of generating an artificial 3D tissue culture of a cancer grown in non-cancerous tissue, comprising the steps of providing an aggregate of pluripotent stem or progenitor cells, culturing and expanding said stem or progenitor cells in a 3D biocompatible matrix, wherein the cells are allowed to differentiate to develop the aggregate into a tissue culture of a desired size; wherein at least a portion of said cells are subjected to carcinogenesis by expressing a oncogene and/or by suppressing a tumor suppressor gene during any of said steps or in the tissue culture, and further comprising the step of allowing said cells with an expressed oncogene or suppressed tumor suppressor to develop into cancerous cells.

The inventive method is also useful in testing unknown genes instead of one or more (known) oncogenes or tumor suppressors. The culture may also be used to test candidate agents for its carcinogenesis potential. Accordingly, the invention also provides a method of screening one or more candidate genes or agents for their effects on carcinogenesis, comprising generating an artificial 3D tissue culture, comprising the steps of providing an aggregate of pluripotent stem or progenitor cells, culturing and expanding said stem or progenitor cells in a 3D biocompatible matrix, wherein the cells are allowed to differentiate to develop the aggregate into a tissue culture of a desired size; wherein at least a portion of said cells are subjected to carcinogenesis by expressing or suppressing the candidate gene or by treating the cells with the candidate agent during any of said steps or in the tissue culture, and further comprising the step of culturing said cells in conditions that allow an expressed or supressed candidate gene to develop into cancerous cells.

The invention further provides an artificial 3D tissue culture, for example an organoid, comprising non-cancerous tissue and cancerous tissue. In the artificial 3D tissue culture, preferably the cancerous tissue overexpresses one or more oncogenes and/or has suppressed (e.g. expression or activity) of one or more tumor suppressors, wherein preferably gene expression of other genes than said oncogene or tumor suppressor is substantially unmodified in the cancerous tissue as compared to the non-cancerous tissue, wherein said tissue (i) is obtainable by a method according to the invention; and/or (ii) comprising a transgene or a construct for suppression of a tumor suppressor at least in cells of the cancerous tissue; and/or (iii) comprising a 3D biocompatible matrix, preferably gel, a collagenous gel, or a hydrogel.

Further provided is a method of testing a candidate compound for carcinogenesis or for its effect on cancer tissue, comprising contacting cells or a tissue in a method of the invention with the candidate compound or contacting a tissue of the invention with the candidate compound and maintaining said contacted tissue in culture, and observing any changes in the tissue as compared to said tissue without contacting by said candidate compound. Likewise, the invention provides exposing the tissue or the cells in the inventive method to a condition instead of contacting it with a candidate compound. Such a condition may be e.g. elevated temperature, limited or increased nutrients or altered redox potential, to which cancer cells may react and exhibit a different behaviour or growth rate.

Further provided is a method of testing a candidate oncolytic virus for carcinogenesis or for its effect on cancer tissue, comprising contacting cells or a tissue in a method of the invention with the candidate oncolytic virus or contacting a tissue of the invention with the candidate oncolytic virus and maintaining said contacted tissue in culture, and observing any changes in the tissue as compared to said tissue without contacting by said candidate oncolytic virus. Likewise, the invention provides exposing the tissue or the cells in the inventive method to a condition instead of contacting it with a candidate oncolytic virus. Such a condition may be e.g. elevated temperature, limited or increased nutrients or altered redox potential, to which cancer cells may react and exhibit a different behaviour or growth rate.

In a further aspect, the invention provides Zika virus for use as a medicament, in particular as an oncolytic virus. In particular provided is Zika virus for use in the treatment of nervous system cancer. Related thereto is a method of treating a nervous system cancer in a patient comprising treating a patient having nervous system cancer with Zika virus to remove said cancer. Further provided is the use of Zika virus in the manufacture of a medicament for the treatment of neuronal cancer. Nervous system cancer or neuronal cancer may e.g. be glioblastoma or neuroblastoma or CNS-PNET (central nervous system primitive neuro-ectodermal tumor).

Further provided is a pharmaceutical composition comprising a replication competent Zika virus and a stabilizer for said virus.

Also provided is a kit suitable for providing a tissue culture according to the invention. The kit may comprise (i) a transfection vector comprising an oncogene transgene or a construct for disruption of a tumor suppressor, (ii) a 3D biocompatible matrix, preferably further comprising (iii) a tissue differentiation agent, a stem cell culturing medium, a nucleofection medium or a combination thereof.

All embodiments of the invention are described together in the following detailed description and all preferred embodiments relate to all embodiments, aspects, methods, tissues and kits alike. E.g. kits or their components can be used in or be suitable for inventive methods. Any component used in the described methods can be in the kit. Inventive tissues are the results of inventive methods or can be used in inventive methods. Preferred and detailed descriptions of the inventive methods read alike on suitability of resulting tissues of the inventions. All embodiments can be combined with each other, except where otherwise stated.

### DETAILED DESCRIPTION OF THE INVENTION

The present invention relates to a method of generating an artificial 3D (three-dimensional) tissue culture of a cancer grown in non-cancerous tissue. Such a 3D tissue culture can be created in vitro and shows all distinguishing characteristics of in vitro cell cultures, such as due to lack of neighbouring obstacles (such as other organs or bones found in vivo) a substantially uniforms shape and/or no directional orientation—except if such have been artificially introduced, as e.g. using directional growth substrates such as disclosed in WO 2017/121754 A1. The produced 3D culture is preferably in all embodiments of the invention an organoid. An organoid is a collection of organspecific cell types that develops from stem cells or organ progenitors and self-organizes through cell sorting and spatially restricted lineage commitment in a manner similar to in vivo (Lancaster and Knoblich, Science 345(6194), 2014: 1247125).

The invention provides 3D tissue cultures that comprise cancerous tissue and thus serve as in vitro models for cancer and cancer development. This allows any research uses such as drug screening or testing reactions of the tissue culture and/or the cancer or non-cancer parts within to environmental influences, such as nutritional or temperature changes or exposure to other agents or compounds. Accordingly, the invention also relates to a method of screening one or more candidate genes or agents for their effects on carcinogenesis or cancer therapy. Said candidate genes or agents may be any such drug or influence or genetic modification, like suppression of one or more suspected tumor suppressors and/or enhancement of expression of one or more suspected oncogenes.

A hallmark of the invention is that the cancerous tissue is grown on or starts growing from non-cancerous tissue. This allows the recapitulation of more in vivo-like effects, like infiltration or invasion and monitoring of early cancerous changes. Accordingly, non-cancerous or pre-cancerous cells are cultured and then carcinogenesis is initiated at a stage of development of the culture of the practitioners choosing, preferably at a stage when progenitor cells have already developed. A progenitor cell is a biological cell that, like a stem cell, has a tendency to differentiate into a specific type of cell, but is already more specific than a stem cell and is pushed to differentiate into its target cell type. Progenitor cells are of the tissue type the organoid is destined to develop into, for example a neuronal progenitor cell. Carcinogenesis is also referred to herein as oncogenesis or tumorigenesis.

As summarized in the background section, previous cultures, including organoid were cultured from cancerous cells, e.g. cells from a patient that a particular type of cancer, like glioblastoma (Huber et al., Cancer Res 2016 76(8): 2465-2477). Such organoids fail to develop the intricate organ structure of normal healthy tissue, including different areas of development or differentiation, as the tumorous mass is the only product of such organoids. Contrary thereto, the invention allows the development of normal tissues with various areas of development or layers of development as can be recapitulated by such 3D tissue cultures or organoids. Upon carcinogenesis initiation, different behaviours or differently differentiated cells can be observed. In particular, like in in vivo situation, not all cell lineages present in the 3D tissue cultures may give rise to a particular type of cancer. This different behaviour of different cell types can be observed by the invention. Accordingly, the invention facilitates tracking and identification of a particular cell lineage that can give rise to a particular cancer in view of the carcinogenesis. According to the invention, carcinogenesis is a modification of the non-cancerous cells of the culture, i.e. introduction of cancerous mutations in the cells of the tissue, not an infiltration by cancerous cells, such as investigated in a metastatic organoid model disclosed in WO 2017/05173 A1. According to the invention, also non-metastatic cancer can be investigated. Accordingly, in a preferment, the cancerous cells of the inventive tissue are non-metastatic, i.e. have no metastasis potential. In other embodiments, the cancerous cells are metastatic. Such metastasis behaviours can e.g. be determined by monitoring cells leaving the 3D tissue culture and being able to form a new tumor or cancer in another organoid. In preferred embodiments, the cancer in non-metalizing or is metastasizing with the proviso that no metastasis of external or internal origin is formed in the 3D tissue culture. “External origin metastasis” means a metastasis from a cell that has not been developed in the 2D tissue culture or aggregate. “Internal origin metastasis” means a metastasis from a cell that has developed in the 2D tissue culture or aggregate; this may be allowed or not.

The inventive method is based on known methods to generate 3D tissue cultures or organoids, such as disclosed in Lancaster, M. A. et al. Nature 501, 373-379 (2013); Lancaster et al., Nature Protocols 9 (10) (2014): 2329-2340; WO 2014/090993 A1; WO 2017/121754 A1; WO 2017 123791 A1; WO 2017 117547 A1; WO 2015/135893 A1 (neuronal and neural organoids); WO 2017/142069 A1 (gastric organoids); WO 2017 115982 A1 (cartilage organoid); WO 2017/059171 A1; WO 2016/183143 A1; WO 2015/184273 A1 (cardiac organoid); WO 2017/049243 A1; WO 2015/130919 A1 (kidney organoid); WO 2016/141137 A1 (vascular organoid); WO 2016/174604 A1 (breast/ductal-lobular organoid); WO 2015/196012 A1 (prostate organoid); WO 2016/061464 A1 (intestinal organoid); WO 2015/183920 A1 (gastric organoid); WO 2014/127170 A1; WO 2012/014076 A2 (liver organoid) (all incorporated herein by reference). Any such organoid generation methods or the resulting organoids can be used according to the invention.

Usually, the inventive method comprises the step of providing an aggregate of pluripotent stem or progenitor cells. Such an aggregate may be developed from a single stem cell, such as an induced pluripotent stem (iPS) cell or an isolated embryonic pluripotent stem cell. For example, the stem cell or progenitor may be a cell isolated from an early stage of an embryo. Such a method does not require the destruction of the embryo. The cell may be developed to an aggregate, also referred to “embryoid body” in the field, as disclosed in the references cited in the above paragraph, especially WO 2014/090993 A1. The aggregate is used as a starting point for the inventive method but of course the invention can also be defined by performing these steps to arrive at the aggregate stage. The following applies to performing said method but also to the resulting aggregate.

The use of iPS cells allows applications in personalized diagnostics and medicine. A cell from a patient, in particular a tumor patient, can be transformed into an iPS cell and used in the inventive methods. Said cell may be a normal, healthy cell from said patient. The inventive carcinogenesis can be used to recapitulate the tumor of the same patient, thereby allowing an investigation of tumorigenesis based on such a tumor cell and its carcinogenic modifications.

For example, a tumor cell from a patient can be examined for aberrant modifications that may lead to tumor development. Such modifications may be mutations or changes in DNA methylation or gene expression. The inventive carcinogenesis can reflect said modification, e.g. by mutating a gene that is modified, aberrantly methylated or expressed in said tumor cell. Assuming that the gene expression is the final effectual cause of tumor development, any such modification can be used to modify a cell according to the carcinogenesis step in the inventive method, as long as gene expression is altered, in particular as reminiscent of the tumor cell from the patient.

If a particular modification has been confirmed by the inventive method as cause of the disease, then said gene may be specifically targeted by individualized medicine, such as by up- or downregulating activity of said gene or its gene product into the direction of the normal activity status. Up- or downregulation can be achieved by conventional means as known in the art, such as by administering a drug or transgene or inhibitory compounds, like inhibitory nucleic acids.

The inventive aggregate can be obtained from culturing pluripotent stem or progenitor cells or a single cell. In preferred embodiments, the aggregate or the cells of the 3D tissue culture are of the same genetic lineage, such as when derived from the same single cell. In principle, the cells may also be totipotent, if ethical reasons allow. A “totipotent” cell can differentiate into any cell type in the body, including the germ line following exposure to stimuli like that normally occurring in development. Accordingly, a totipotent cell may be defined as a cell being capable of growing, i.e. developing, into an entire organism. The cells used in the methods according to the present invention are preferably not totipotent, but (strictly) pluripotent.

In a particular preferred embodiment, the cells of the present invention (including all further embodiments related thereto), are human cells or non-human cells, e.g. primate cells. The inventive cells are usually eukaryotic cells. Further non-human animals as origin of the cells are mouse, cat, dog, hamster, rodent, rat, cow, horse, sheep, etc.

A “pluripotent” stem cell is not able of growing into an entire organism, but is capable of giving rise to cell types originating from all three germ layers, i.e., mesoderm, endoderm, and ectoderm, and may be capable of giving rise to all cell types of an organism. Pluripotency can be a feature of the cell per see, e.g. in certain stem cells, or it can be induced artificially. E.g. in a preferred embodiment of the invention, the pluripotent stem cell is derived from a somatic, multipotent, unipotent or progenitor cell, wherein pluripotency is induced. Such a cell is referred to as induced pluripotent stem cell herein. The somatic, multipotent, unipotent or progenitor cell can e.g. be used from a patient, which is turned into a pluripotent cell, that is subject to the inventive methods. Such a cell or the resulting tissue culture can be studied for abnormalities, e.g. during tissue culture development according to the inventive methods.

A “multipotent” cell is capable of giving rise to at least one cell type from each of two or more different organs or tissues of an organism, wherein the said cell types may originate from the same or from different germ layers, but is not capable of giving rise to all cell types of an organism.

In contrast, a “unipotent” cell is capable of differentiating to cells of only one cell lineage.

A “progenitor cell” is a cell that, like a stem cell, has the ability to differentiate into a specific type of cell, with limited options to differentiate, with usually only one target cell. A progenitor cell is usually a unipotent cell, it may also be a multipotent cell.

With decreasing differentiation capabilities, stem cells differentiate in the following order: totipotent, pluripotent, multipotent, unipotent. For example, during development of the inventive organoid, stem cells differentiate from pluripotent (also totipotent cells are possible) into multipotent neural stem cells, further into unipotent stem cells of a tissue type and subsequently into non-stem tissue cells. Tissue cells may e.g. be neuronal cells or neuroepithelial cells, such as glial cells.

The aggregate or tissue is preferably treated with a differentiation factor in order to initiate differentiation into a specific tissue type of interest. Alternatively, the aggregate can already be induced for particular tissue type differentiation, e.g. by treating the cells during its generation. Accordingly, the aggregate preferably develops into a differentiated tissue type of interest. Since cancer may befall any tissue, any tissue type is also possible for the invention. Likewise, culturing is known for virtually any tissue type, including the generation of organoids from any tissue. In preferred embodiment, the aggregate or inventive tissue comprises, is or develops into (“tissue fate”) neuronal, gastric, connective, cartilage, bone, bone marrow, cardiac, kidney, vascular, breast or ductal-lobular, retinal, prostate, intestinal, gastric, lung, endothelium or liver tissue. The progenitor cell may be of any of these tissues or may be destined for development into any of these tissues. In particular preferred is neuronal tissue, in particular cerebral tissue. The differentiation factor that is administered to the cells or aggregate is for differentiation into any such a tissue. The aggregate or tissues may comprise any stem or progenitor cell for such a tissue that has undergone tissue specific differentiation. Preferably the tissues comprise cells selected from neuronal or neurogenic, adipogenic, myogenic, tenogenic, chondrogenic, osteogenic, ligamentogenic, dermatogenic, hepatic, or endothelial cells. In some cases, also combinations are possible, e.g. organ cells (e.g. neuronal, myogenic, hepatic) with cells that would develop into supporting tissues (e.g. endothelial, adipogenic, ligamentogenic cells). In the methods, differentiation may be initiated by commonly known tissue specific growth or differentiation factors, also called, differentiation-inducing agents. Such are e.g. known in the art and are e.g. disclosed in WO 2009/023246 A2, WO 2004/084950 A2 and WO 2003/042405 A2. Further, the differentiating or growth factor can be a bone morphogenetic protein, a cartilage-derived morphogenic protein, a growth differentiation factor, an angiogenic factor, a platelet-derived growth factor, a vascular endothelial growth factor, an epidermal growth factor, a fibroblast growth factor, a hepatocyte growth factor, an insulin-like growth factor, a nerve growth factor, a colony- stimulating factor, a neurotrophin, a growth hormone, an interleukin, a connective tissue growth factor, a parathyroid hormone-related protein, (e.g. disclosed in WO 2004/084950 A2). These factors/agents are commercially available and need no further description. Of course, such factors/agents for any one of the above tissue types may be included in the inventive kit. Preferably, neural, neuronal or neurogenic differentiation factors are used in the method or provided in the kit and preferably neural, neuronal or neurogenic cells are present in the inventive tissue. In a preferred method of the invention, the pluripotent stem or progenitor cells are differentiated into neural or neuronal cells and/or the tissue is developed into an organoid. In case of neuronal or neural 3D tissue cultures with cancerous tissue, the invention is also referred to as brain neoplastic organoids herein.

The aggregate or tissue may comprise progenitor cells, such as a stem cell, to any tissue, especially those described above. The progenitor cell is preferably selected from the group consisting of a totipotent stem cell, pluripotent stem cell, multipotent stem cell, mesenchymal stem cell, neural stem cell, hematopoietic stem cell, pancreatic stem cell, cardiac stem cell, embryonic stem cell, embryonic germ cell, neural stem cell, especially a neural crest stem cell, kidney stem cell, hepatic stem cell, lung stem cell, hemangioblast cell, and endothelial progenitor cell. The pluripotent cell used in the method or the progenitor cell can be derived from a de-differentiated chondrogenic cell, myogenic cell, osteogenic cell, tendogenic cell, ligamentogenic cell, adipogenic cell, neurogenic cell or dermatogenic cell. A neuronal stem cell or progenitor cells may be differentiated to astrocytes and/or oligodendrocytes and optionally further to glia cells. At any such stage, carcinogenesis can take place. Preferably carcinogenesis is at a stage of neuroectoderm, e.g. when the aggregate or tissue comprises neuroectoderm.

Differentiation can be achieved by contacting cells with a tissue specific growth or differentiation factor. The cells may then develop into the desired tissue. Such a tissue specific growth or differentiation factor may be a neuronal or neurogenic, myogenic, tenogenic, chondrogenic, or osteogenic differentiation factor, etc. Especially preferred is a neuronal differentiation factor. This will determine the development into the respective type of cellular tissue in later development. The cells will thereby transit from pluripotent to multipotent cells. Other tissue types shall then be not or only by a return to a pluripotent status be possible again. Usually not all cells are differentiated to the selected tissue type. It usually sufficient when about 30% or more or at least 40% or at least 50%, or at least 60% or at least 70% or at least 80% of the cells initiate differentiation towards the selected tissue type and transform to reduce their differentiation potential by multipotent cell with the respective tissue destiny (%-values as fractions of the cell amount). Of course, this differentiation destiny only applies for the cells that are not returned to an un- or less differentiated state by use of artificial growth and dedifferentiation stimuli. Clearly, even somatic cells can be returned to a pluripotent cell and this is not meant when defining a differentiated state herein. Preferably, no factors are introduced to the cells that would return the cells to pluripotent cells.

The aggregate of pluripotent stem or progenitor cells is cultured and expanded in a 3D biocompatible matrix, wherein the cells are allowed to differentiate to develop the aggregate into a tissue culture of a desired size and/or further advanced differentiation status. This advanced may be an increased number of different tissue-specific differentiation states, such as for example cells of at least two different progenitors and tissue-specific (e.g. neural or neuronal) differentiation layers. In case of neuronal tissue, it is preferred that at least one progenitor layer comprises outer radial glia cells, cells of an outer or extra cortical subventricular zone and/or cells of a cortical inner fiber layer.

In the inventive method of creating a cancer-containing tissue, at least a portion of said stem or progenitor cells are subjected to carcinogenesis. “Stem or progenitor cells” for carcinogenesis of course also includes any descendent cell, including cells into which these stem or progenitor cells have differentiated in the aggregate or 3D tissue culture. As said above, carcinogenesis can be initiated at any stage of development of the aggregate or 3D tissue, which includes any stage of development of the cells, wherein preferably, tissue specificity or a tissue fate has already been initiated when carcinogenesis starts. Carcinogenesis is achieved by expressing an oncogene and/or by suppressing a tumor suppressor gene during any of the method steps (e.g. in cells of the aggregate before or during 3D matrix culturing) or in the tissue culture. Said cells with an expressed oncogene or suppressed tumor suppressor gene are allowed to develop into cancerous cells.

In the inventive method of screening (i.e. testing) one or more candidate genes or agents for their effects on carcinogenesis, at least a portion of said stem or progenitor cells including their descendants are subjected to potential carcinogenesis by expressing or suppressing the candidate genes or by treating the cells with the candidate agents during any of the method steps (e.g. in cells of the aggregate before or during 3D matrix culturing) or in the tissue culture. The cells or tissue is cultured in conditions that allow a cell with an expressed or supressed candidate genes or subject to the agent to develop into cancerous cells. Such conditions may be normal culturing conditions usually used for 3D tissue cultures or organoids. These include culturing in media with nutrients and at a suitable temperature and pressure for the cells, non-cancerous or cancerous alike.

Carcinogenesis (or tumorigenesis) is an artificial step in the inventive method. In involves the expression or overexpression of an oncogene, also referred to as “tumor gene” or the reduced or inhibited expression of a tumor suppressor gene, or both, or combinations of more than one such expressions/overexpressions of an oncogene and/or the reduced or inhibited expressions of tumor suppressor genes. This leads to the development of a cancer in cells of the 3D artificial tissue culture—that is, at least a portion of said cells, not necessarily all cells.

Hundreds of oncogenes and tumor suppressor genes are known in the art. Such genes, have been collected in data bases such as “Cancer Gene Census” at cancer.sanger.ac.uk/census/ (Futreal et al. Nature Reviews Cancer 4, 177-183 (2004)). Any known oncogene or tumor suppressor gene can be used in the inventive method, in particular to test its effect on carcinogenesis in the inventive tissue or organoid.

Preferably the oncogene, tumor suppressor or candidate gene are selected from cancer genes selected from the group consisting of ABL1, ABL2, AF15Q14, AF1Q, AF3p21, AF5q31, AKT, AKT2, ALK, ALO17, AML1, API, APC, ARHGEF, ARHH, ARNT, ASPSCR1, ATIC, ATM, AXL, BCL10, BCL11A, BCL11B, BCL2, BCL3, BCL5, BCL6, BCL7A, BCL9, BCR, BHD, BIRC3, BLM, BMPR1A, BRCA1, BRCA2, BRD4, BTG1, CBFA2T1, CBFA2T3, CBFB, CBL, CCND1, c-fos, CDH1, c-jun, CDK4, c-kit, CDKN2A-p14ARF, CDKN2A-p16INK4A, CDX2, CEBPA, CEP1, CHEK2, CHIC2, CHN1, CLTC, c-met, c-myc, COL1A1, COPEB, COX6C, CREBBP, c-ret, CTNNB1, CYLD, D10S170, DDB2, DDIT3, DDX10, DEK, EGFR, EIF4A2, ELKS, ELL, EP300, EPS15, erbB, ERBB2, ERCC2, ERCC3, ERCC4, ERCC5, ERG, ETV1, ETV4, ETV6, EVI1, EWSR1, EXT1, EXT2, FACL6, FANCA, FANCC, FANCD2, FANCE, FANCF, FANCG, FEV, FGFR1, FGFR1OP, FGFR2, FGFR3, FH, FIP1L1, FLI1, FLT3, FLT4, FMS, FNBP1, FOXO1A, FOXO3A, FPS, FSTL3, FUS, GAS7, GATA1, GIP, GMPS, GNAS, GOLGA5, GPC3, GPHN, GRAF, HEI1O, HER3, HIP1, HIST1H4I, HLF, HMGA2, HOXA11, HOXA13, HOXA9, HOXC13, HOXD11, HOXD13, HRAS, HRPT2, HSPCA, HSPCB, hTERT, IL21R, IRF4, IRTA1, JAK2, KIT, KRAS2, LAF4, LASP1, LCK, LCP1, LCX, LHFP, LMO1, LMO2, LPP, LYL1, MADH4, MALT1, MAML2, MAP2K4, MDM2, MECT1, MEN1, MET, MHC2TA, MLF1, MLH1, MLL, MLLT1, MLLT10, MLLT2, MLLT3, MLLT4, MLLT6, MLLT7, MLM, MN1, MSF, MSH2, MSH6, MSN, MTS1, MUTYH, MYC, MYCL1, MYCN, MYH11, MYH9, MYST4, NACA, NBS1, NCOA2, NCOA4, NF1, NF2, NOTCH1, NPM1, NR4 A3, NRAS, NSD1, NTRK1, NTRK3, NUMA1, NUP214, NUP98, NUT, OLIG2, p53, p27, p57, p16, p21, p73, PAX3, PAX5, PAX7, PAX8, PBX1, PCM1, PDGFB, PDGFRA, PDGFRB, PICALM, PIM1, PML, PMS1, PMS2, PMX1, PNUTL1, POU2AF1, PPARG, PRAD-1, PRCC, PRKAR1A, PRO1073, PSIP2, PTCH, PTEN, PTPN11, RAB5EP, RAD51L1, RAF, RAP1GDS1, RARA, RAS, Rb, RB1, RECQL4, REL, RET, RPL22, RUNX1, RUNXBP2, SBDS, SDHB, SDHC, SDHD, SEPT6, SET, SFPQ, SH3GL1, SIS, SMAD2, SMAD3, SMAD4, SMARCB1, SMO, SRC, SS18, SS18L1, SSH3BP1, SSX1, SSX2, SSX4, Stathmin, STK11, STL, SUFU, TAF15, TALI, TAL2, TCF1, TCF12, TCF3, TCL1A, TEC, TCF12, TFE3, TFEB, TFG, TFPT, TFRC, TIF1, TLX1, TLX3, TNFRSF6, TOP1, TP53, TPM3, TPM4, TPR, TRA, TRB, TRD, TRIM33, TRIP11, TRK, TSC1, TSC2, TSHR, VHL, WAS, WHSC1L18, WRN, WT1, XPA, XPC, ZNF145, ZNF198, ZNF278, ZNF384, ZNFN1A1, and especially preferred from MYC, CDKN2A, CDKN2B, EGFR, EGFRvIII, NF1, PTEN, p53 (TP53) or combinations thereof. Also preferred are PTEN, ATM, ATR, EGFR, ERBB2, ERBB3, ERBB4, Notch1, Notch2, Notch3, Notch4, AKT, AKT2, AKT3, HIF, HIF1a, HIF3a, Met, HRG, Bcl2, PPAR alpha, PPAR gamma, WT1 (Wilms Tumor), FGF Receptor Family members (members 1, 2, 3, 4, 5), CDKN2a, APC, RB (retinoblastoma gene), MEN1, VHL, BRCA1, BRCA2, AR (androgen receptor), TSG101, IGF, IGF receptor, Igf1, Igf2, Igf 1 receptor, Igf 2 receptor, Bax, Bcl2, caspase family (members 1, 2, 3, 4, 6, 7, 8, 9, 12) Kras, Apc. Further and preferred cancer genes that can be used according to the invention are one or more of the following genes: CDKN2A, CDKN2B, CDKN2C, NF1, PTEN, p53 (TP53), ATRX, RB1, CDK4, CDK6, MDM2-B, EGFR, EG-FRvIII, PDGFRA, H3F3A (preferably a K27M or G34R alteration), MYC, SMARB1, PTCH1, CTNNB1, MET, RTK, FGFR1, FGFR2, FGFR3, PI3-kinase, PIK3CA, PIK3R1, PIK3C2G, PIK3CB, PIK3C2B, PIK3C2A, PIK3R2, PTEN, BRAF, MDM2, MDM4, MDM1, IDH1, IDH2; or combinations thereof such as CDKN2A and CDKN2B, CDKN2A and CDKN2B and EGFR, CDKN2A and CDKN2B and EGFRvIII, CDKN2A and CDKN2B and EGFR and EGFRvIII, CDKN2A and CDKN2B and PTEN, CDKN2A and CDKN2B and p53, CDKN2A and CDKN2B and PDGFRA, EGFR and CDK4, EGFRvIII and CDK4, EGFR and EGFRvIII and CDK4, MDM2-B and CDK4, NF1 and PTEN and p53, EGFRvIII and CDKN2A and PTEN, H3F3A and ARTX and p53. The gene abbreviations or gene names are used in the art and full gene names are summarized in gene databases such as the NCBI database or the EPI database. The database GeneCards (www.genecards.org/) collects information from various databases and provides accumulated summaries. Gene Cards is the preferred database to procure further information from these genes. Preferred combinations are (i) CDKN2A, CDKN2B, EGFR, and EGFRvIII, (ii) NF1, PTEN and p53, or (iii) EGFRvIII, CDKN2A and PTEN, or (iv) MYC. Such combinations are for example (i) CDKN2A⁻/CDKN2B⁻/EGFR^{OE}/EGFRvIII^{OE}, (ii) NF1⁻/PTEN⁻/p53⁻, or (iii) EG-FRvIII^{OE}/CDKN2A⁻/PTEN⁻, or (iv) MYC^{OE}, with a raised minus sign (“⁻”) indicating a reduced or inhibited expression and raised letters OE (“^{OE}”) indicating an overexpression. The carcinogenic mutation in these genes as used in the invention is preferably a change as found in in vivo patients or cancerous cells. Such mutations can be easily identified or have already been identified by comparison of the genes with wild-type nucleic acid sequences in healthy cells. Carcinogenic are known from various publications or the data bases mentioned herein.

Preferably an oncogene is selected from ras, raf, Bcl-2, Akt, Sis, src, Notch, Stathmin, mdm2, abl, hTERT, c-fos, c-jun, c-myc, erbB, HER2/Neu, HER3, c-kit, c-met, c-ret, flt3, API, AMLl, axl, alk, fms, fps, gip, lck, MLM, PRAD-1, and trk. Preferably a tumor suppressor genes is selected from p53, Rb, PTEN, BRCA-1, BRCA-2, APC, p57, p27, p16, p21, p73, p14ARF, Chek2, NF1, NF2, VHL, WRN, WT1, MEN1, MTS1, SMAD2, SMAD3, and SMAD4.

In the inventive screening methods, a candidate gene of choice may be mutated or otherwise, mutations in random genes may be introduced, such as by non-specific mutagenesis, like irradiation or chemical carcinogenesis or by oncogenic virus exposure, such as a retrovirus or a DNA virus, e.g. a papilloma virus. Such altered genes may be identified by genetic analysis.

In particular preferred are carcinogenic mutations found in neuronal tumors, in particular in case the cells of the aggregate or 3D tissue culture comprise neural cells. Such gen alterations are known in the art and published in the above-mentioned databases or in scientific literature, such as in Brennan et al. Cell 2013; 155(2): 462-477 or McLendon et al., 2008, Nature, 455, 1061-8.

The selection of oncogenes and/or tumor suppressor genes may also be selected according to the genotype in clinical cancer, found in a patient. Accordingly, the invention also provides method of detecting aberrantly expressed genes in a cancer cell of a patient and expressing or suppressing such genes (according to the expression pattern found in the patient), as above, in the cells of the inventive aggregate or 3D tissue culture. The detection of aberrantly expressed genes in the patient may be in comparison with healthy cells of the patient or with cells of other healthy individuals of the same species as controls, preferably, wherein said comparison cells/control cells are also of the same tissue or differentiation type as the cancer cells that is analysed. An aberrant expression may be a deviation in expression level of at least 25%, preferably at least 30%, at least 50% or at least 75% decrease or increase (all %-in mol.-%). Other kinds of carcinogenic mutations are in addition or alternatively to expression level changes in the coding sequence and may include loss or gain of function mutations. Loss or gain of function may be a change in gene product activity of at least 25%, preferably at least 30%, at least 50% or at least 75% decrease or increase (all %-in enzymatic activity unit U-% or katal-%). Expression levels and activities all relates to the wild-type expression or activity of said gene/gene product. In particular embodiments, tumor suppressor genes are prevented by knock-out mutations. In particular embodiments, oncogenes may be introduced (if they do not exist in healthy cells) or have an at least 2-fold, preferably at least 4-fold expression/activity as compared to the control (as above, mol.-% or enzymatic unit, katal).

Methods to introduce such mutations are well-known in the art, and include knock-out or knock-down methods or mutagenesis by e.g. CRISPR-Cas or homologous recombination with a transgene. For this purpose, genetic material able to cause said mutation is introduced in the cells. Genetic constructs may be used to introduce such genetic material into the cells. Constructs may e.g. expression vectors, integration vectors, transposons or a virus.

Example methods for carcinogenic mutation by introduction of a construct or other genetic material are transfection or transduction. Transfection of cells typically involves opening transient pores or holes in the cell membrane to allow the uptake of material (U.S. Pat. No. 7,732,175 B2). Transfection can be carried out using calcium phosphate (i.e. tricalcium phosphate), by electroporation, by cell squeezing or by mixing a cationic lipid with the material to produce liposomes which fuse with the cell membrane and deposit their cargo inside. There are various methods of introducing foreign DNA into a eukaryotic cell: some rely on physical treatment (electroporation, cell squeezing, nanoparticles, magnetofection); others rely on chemical materials or biological particles (viruses) that are used as carriers. Non-viral methods include physical methods such as electroporation, microinjection, gene gun, impalefection, hydrostatic pressure, continuous infusion, cell squeezing, optical laser transfection, gene gun transfection (particle bombardment), magnetofection, and sonication (sonoporation) and chemical, such as lipofection, which is a lipid-mediated DNA-transfection process utilizing liposome vectors, calcium phosphate transfection, dendrimer transfection, polycation transfection, FuGENE transfection. It can also include the use of polymeric gene carriers (polyplexes). These methods may be combined with each other or other assisting techniques, such as a heat shock.

Virus-mediated gene delivery utilizes the ability of a virus to inject its DNA inside a host cell. A gene that is intended for delivery is packaged into a replication-deficient viral particle. Viruses used to date include retrovirus, lentivirus, adenovirus, adeno-associated virus and herpes simplex virus. Transduction with viral vectors can be used to insert or modify genes in mammalian cells. E.g., a plasmid is constructed in which the genes to be transferred are flanked by viral sequences that are used by viral proteins to recognize and package the viral genome into viral particles. This plasmid is inserted (usually by transfection) into a producer cell together with other plasmids (DNA constructs) that carry the viral genes required for formation of infectious virions. In these producer cells, the viral proteins expressed by these packaging constructs bind the sequences on the DNA/RNA (depending on the type of viral vector) to be transferred and insert it into viral particles. Preferably, none of the plasmids used contains all the sequences required for virus formation, so that simultaneous transfection of multiple plasmids is required to get infectious virions. Also preferred, only the plasmid carrying the sequences to be transferred contains signals that allow the genetic materials to be packaged in virions, so that none of the genes encoding viral proteins are packaged. Viruses collected from such production cells are then applied to the cells of the 3d tissue culture or the aggregate to be altered.

The introduction of conditional mutations is possible by using suitable conditional mutation vectors, e.g. comprising a reversible gene trap. Conditional mutations preferably facilitate reversible mutations, which can be reversed to a gene-active or inactive, respectively, state upon stimulation, e.g. as in the double-Flex system (WO 2006/056615 A1; WO 2006/056617 A1; WO 2002/88353 A2; WO 2001/29208 A1). Mutations can either be random or site-directed at specific genes. Thus, in some embodiments of the invention, reversible mutations are introduced into the pluripotent stem cells, either by random (forward) or site directed (reverse) mutagenesis. Suitable vectors comprising insertion cassette with a reversible mutation. Mutations can be switched on or off at any stage of the inventive method. Preferred mutations are non-reversible and inheritable to the cancer or precancer cell progeny cells.

Genetic material capable of carcinogenesis may encode any agonist of an oncogene or inhibitor (or antagonist) of a tumor suppressor gene. Such genetic elements may be expression vectors, expressing integration vectors or knock-in vector (agonists) or inhibitory nucleic acids, knock-out or knock-down vector (inhibitors). Exemplary inhibitors of tumor suppressor genes include antisense oligonucleotides or inhibitory RNA molecules, such as small interfering RNAs (siRNAs), short hairpin RNAs (shRNAs), microRNAs (miRNAs), Piwi-interacting RNAs (piRNAs), and small nuclear RNAs (snRNAs), and Clustered Regularly Inter-spaced Short Palindromic Repeats (CRISPR) interference (CRISPRi) systems comprising guide crRNAs and Cas protein that downregulate expression of one or more tumor suppressor genes. Cas may be a nuclease-deficient Cas (e.g., dCas9). Such inhibitors may again be encoded by expression systems of the inhibitor, e.g. as the oncogene with an expression vector or expressing integration vector.

Any type of promoter can be operably linked to a target nucleic acid sequence. Examples of promoters include, without limitation, tissue-specific promoters, constitutive promoters, inducible promoters, and promoters responsive or unresponsive to a particular stimulus.

Targeted editing tools can be used for both over or reduced/inhibited expression, e.g. by enhancing a promoter of an oncogene, homologous recombination (knock-in) and introduction of a gene for an oncogene, or disruption, ablation or inhibited expression of a tumor suppressor gene. Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is a REGEN. tracrRNA is another such tool. These are examples of targeted nuclease systems: these systems have a DNA-binding member that localizes the nuclease to a target site. The site is then cut by the nuclease. TALENs and ZFNs have the nuclease fused to the DNA-binding member.

Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at nor near an intended site. Certain embodiments are applicable to all such systems without limitation; including, embodiments that minimize nuclease re-cleavage, embodiments for making indels with precision at an intended residue, and placement of the allele that is being introgressed at the DNA-binding site. Methods of oncogenesis (=carcinogenesis) using the CRISPR-Cas system are for example disclosed in WO 2014/204723 A1 (incorporated herein by reference) and various other documents. Any such method can be used according to the invention.

For example, as shown in the example section, which is provided for illustration only and not to limit the invention, a combined transposon-mediated insertion with CRISPR/Cas9-mediated genome editing was used to model human brain tumorigenesis in cerebral organoids. By screening multiple combinations of gain- and loss-of-function mutations found in cancer patients (McLendon et al., 2008, Nature, 455, 1061-8), it was demonstrated that the growth of large, xeno-transplantable tumors can be classified as central nervous system primitive neuroectodermal tumor (CNS-PNET) or glioblastoma (GBM) by marker expression and transcriptome analysis. The approach initiates the transformation of tumors carrying a specific set of driver mutations in the genetic background of any patient, which allows the potential targeted drug testing in a personalized manner. Finally, the newly developed 3D brain tumor models were used to screen cancer medication and to demonstrate the oncolytic activity of the Zika flavovirus, thereby establishing its potential suitability for brain tumor therapy. It was demonstrated that these brain tumor models can be used to evaluate drug efficacy on tumors with specific DNA aberrations.

The inventive method further comprises the step of culturing and expanding said stem cells in a 3D biocompatible matrix. In this step, the cells are allowed to differentiate and to develop into a tissue culture of a desired size. As recapitulated above with regard to references disclosing various organoids, such a step is generally known and e.g. disclosed in WO2014/090993 and WO2017/121754. The cells of the aggregate are placed in said 3D biocompatible matrix preferably in form of said aggregate itself, i.e. without isolating cells from the aggregate.

Growth of the tissues to form the 3D tissue culture may be performed as known in the art for tissue culturing in a 3D (three dimensional) matrix. A 3D matrix is distinct from 2D cultures, such as 2D cultures in a dish on a flat surface. A “3D culture” means that the culture can expand in all three dimensions without being blocked by a one-sided wall (such as a bottom plate of a dish). Such a culture is preferably in suspension. The 3D biocompatible matrix may be a gel, especially a rigid stable gel, which results in further expansion of growing cell culture/tissue and differentiation. A suitable 3D matrix may comprise collagen. More preferably the 3D matrix comprises extracellular matrix (ECM) or any component thereof selected from collagen, laminin, entactin, and heparin-sulfated proteoglycan or any combination thereof. Extra-cellular matrix may be from the Engelbreth-Holm-Swarm tumor or any component thereof such as laminin, collagen, preferably type 4 collagen, entactin, and optionally further heparan-sulfated proteoglycan or any combination thereof. Such a matrix is Matrigel. Matrigel is known in the art (U.S. Pat. No. 4,829,000) and has been used to model 3D heart tissue previously (WO 01/55297 A2) or neuronal tissue (WO 2014/090993). Preferably the matrix comprises laminin, collagen and entactin, preferably in concentrations 30%-85% or 50%-85%, laminin, 3%-50% collagen and sufficient entactin so that the matrix forms a gel, usually 0.5%-10% entactin. Laminin may require the presence of entactin to form a gel if collagen amounts are insufficient for gel forming. Even more preferred, the matrix comprises a concentration of at least 3.7 mg/ml containing in parts by weight about 30%-85% laminin, 5%-40% collagen IV, optionally 1%-10% nidogen, optionally 1%-10% heparan sulfate proteoglycan and 1%-10% entactin. Matrigel's solid components usually comprise approximately 60% laminin, 30% collagen IV, and 8% entactin. All %-values given for the matrix components are in wt.-%. Entactin is a bridging molecule that interacts with laminin and collagen. Such matrix components can be added in step r). These components are also preferred parts of the inventive kit. The 3D matrix may further comprise growth factors, such as any one of EGF (epidermal growth factor), FGF (fibroblast growth factor), NGF, PDGF, IGF (insulin-like growth factor), especially IGF-1, TGF-β, tissue plasminogen activator. The 3D matrix may also be free of any of these growth factors.

In general, the 3D matrix is a 3D structure of a biocompatible matrix. It preferably comprises collagen, gelatin, chitosan, hyaluronan, methylcellulose, laminin and/or alginate. The matrix may be a gel, in particular a hydrogel. Organo-chemical hydrogels may comprise polyvinyl alcohol, sodium polyacrylate, acrylate polymers and copolymers with an abundance of hydrophilic groups. Hydrogels comprise a network of polymer chains that are hydrophilic, sometimes found as a colloidal gel in which water is the dispersion medium. Hydrogels are highly absorbent (they can contain 90 wt.-% water or more) natural or synthetic polymers. Hydrogels also possess a degree of flexibility very similar to natural tissue, due to their significant water content. It is possible that the 3D matrix, or its components, especially ECM or collagen, still remains in the produced 3D tissue culture. Preferably the 3D matrix is a collagenous matrix, preferably it contains type I and/or type IV collagen. In particular preferred, the 3D biocompatible matrix is a collagenous gel or a collagenous hydrogel. Preferably, said aggregate of cells and/or the 3D matrix are cultured in a suspension culture. A suspension culture prevents contacts to solid walls of a cultivation vessel and allows the 3D tissue culture during formation to expand in all directions uniformly. The 3D tissue culture may be formed without contacts to such a solid wall or without regions of halted expansion due to contact to such a wall.

In summary of the above, carcinogenesis is preferably performed after the pluripotent stem cells have been stimulated for tissue-specific differentiation, such as neural differentiation. For example, carcinogenesis is before expanding said stem cells in a 3D biocompatible matrix. Carcinogenesis may be a recombinant modification of said genes, preferably by introduction of a transgene for expression of the oncogene or a gene inhibition construct for suppression of the tumor suppressor. The transgene or construct may be introduced into cells by nucleofection such as electroporation.

In a further preferment of the invention the cancerous cells are labelled with a marker, preferably a marker gene. Possible markers or labels are reporter genes such as fluorescent proteins, preferably GFP (green fluorescent protein), enhanced green fluorescent protein (eGFP), d2EGFP, CFP (cyan fluorescent protein), YFP (yellow fluorescent protein), RFP (drFP583; also red fluorescent protein), BFP (blue fluorescent protein), smURFP (Small ultra red fluorescent protein), HcRed, DsRed, DsRed monomer, ZsGreen, AmCyan, ZsYellow enhanced blue fluo-rescent protein (eBFP), enhanced yellow fluorescent protein (eYFP), GFPuv, enhanced cyan fluorescent protein (eCFP), far red Reef Coral Fluorescent Protein; β-galactosidase; luciferase; a peroxidase, e.g. horse radish peroxidase; alkaline phosphatases, e.g., SEAP, and glucose oxidase, any cell sur-face marker such as Thy1.1.

Another type of marker is an enzymatic label. “Enzymatic label” means an enzyme that converts a substrate to a detectable product. Suitable label enzymes for use in the present invention include, but are not limited to, galactosidase, horseradish peroxidase, luciferases, e.g., fire fly and renilla luciferase, alkaline phosphatases, e.g., SEAP, and glucose oxidase. The presence of the marker can be determined through the enzyme's catalysis of substrate into an identifiable product.

Other markers are detectable proteins, in particular cell surface proteins. Surface proteins can be detected by molecular interaction with a binding partner through chemical or physical interaction. A surface protein may be any partner in a “binding pair”. Binding pairs are molecules that interact with each other through binding. “Partner of a binding pair” means one of a first and a second moiety, wherein the first and the second moiety have a suitable binding affinity for each other to detect the pair with its members bound to each other. Suitable binding pairs for use in the invention include, but are not limited to, antigens/antibodies. Preferably, the cancer cells express a tumor antigen that can be detected. Such a tumor antigen may be one of the oncogenes that is artificially expressed as discussed above.

Such a marker can be introduced together with the carcinogenic elements as described above or separate from said elements. Preferred in all embodiments is the introduction together with the carcinogenesis in order to track treated cells. In any case, said labelling with a marker to identify cancerous cells. Accordingly, the invention also comprises the step of identifying cancerous cells in said tissue culture. Said identifying step is preferably performed by identifying the marker. Such methods of identification are well known in the art and include cell sorting (e.g. FACS—fluorescence-activated cell sorting), immunoassays, marker photo detection, magnetic separation etc. Preferably, the marker is a genetic marker that can be passed on to progeny cells of the labelled cells. A labelled cell may be a cell destined for carcinogenesis, it may or may not be a cancer cell already. Preferred markers are different than the oncogenes.

An artificial 3D tissue culture obtainable by any one of the above described and below described methods and preferred embodiments, having accordingly bestowed characteristics, forms also part of the invention. Producing such a 3D tissue culture is usually a step in the inventive method. In addition to the above characteristics, the 3D tissue culture may comprise non-cancerous tissue and cancerous tissue. The cancerous tissue overexpresses an oncogene and/or has suppressed expression of a tumor suppressor as mentioned above, preferably in combination with a marker gene that allows detection. The cancerous genes usually have the same genetic background as the non-cancerous cells, i.e. are from the same source original progenitor cells, e.g. pluripotent stem cells. Accordingly, genes other than said oncogene or tumor suppressor are preferably substantially unmodified in the cancerous tissue as compared to the non-cancerous tissue. Furthermore, said tissue (i) is obtainable by a method according to the invention; and/or (ii) comprising a transgene or a construct for suppression of a tumor suppressor at least in cells of the cancerous tissue; and/or (iii) comprising a 3D biocompatible matrix, preferably gel, a collagenous gel, or a hydrogel as disclosed above. The 3D tissue culture may be an organoid or have any one of an organoid's characteristics, such as they 1. contain multiple organ-specific cell types, i.e. the cells have different differentiation types depending on the general organ selected for differentiation (e.g. neural progenitor cells may further differentiate into forebrain cells, cells of cells of dorsal-lateral ganglionic eminence and caudal ganglionic eminence identity, cells of ventral-medial ganglionic eminence identity, cells of dorsal cortex identity, etc., in general of any subdifferentiation as mentioned above); 2. are capable of recapitulating some specific function of the organ (eg. excretion, filtration, neural activity, contraction); 3. are grouped together and spatially organized similar to an organ. Organoid formation recapitulates both major processes of self-organization during development: cell sorting out and spatially restricted lineage commitment. This self-organization and differentiation according to particular tissue parts reminiscent of in vivo development is found in the inventive 3D tissue culture. Of course, such a natural development is found in the non-cancerous cells. Cancerous cells may differ from natural tissue or organ development and exhibit a cancerous/tumor phenotype, such as uncontrolled growth and in severe cancer case invasion of non-cancerous tissue parts. Preferably, the tissue comprises cells of a cancerous or proliferative central nervous system disorder, in particular preferred glioblastoma, neuroblastoma or CNS-PNET (central nervous system primitive neuro-ectodermal tumor) as further described herein.

In preferred embodiments of the invention, the 3D tissue culture is grown to a size or has a size of at least 100 μm, preferably at least 150 μm, especially preferred at least 200 μm. “Size” refers to the longest dimension in 3d space. Preferably the 3D tissue culture is globular in shape, in particular with the shortest dimension being not less than 20% of the longest dimension, in particular not less than 30% or not less than 40% of the longest dimension. Preferably the volume of the 3D tissue culture is at least 1×10⁶ μm³, in particular preferred at least 2×10⁶ μm³, at least 4×10⁶ μm³, at least 6×10⁶ μm³, at least 8×10⁶ μm³, at least 10×10⁶ μm³, at least 15×10⁶ μm³ and/or sizes of at least 250 μm, especially preferred at least 350 μm.

The 3D tissue culture is usually of a size of at most 10 mm, preferably of at most 5 mm, of at most 2 mm, of at most 1250 μm or at most 800 μm, e.g. with volumes of at most at most 4200 mm³, at most 2400 mm³, at most 1200 mm³, at most 800 mm³, at most 400 mm³, at most 100 mm³, at most 50 mm³, at most 8 mm³, at most 2 mm³, or at most at most 1 mm³. In some embodiments, the 3D tissue culture may be larger with a size of at most 15 mm, preferably of at most 10 mm or at most 5 mm, e.g. with volumes of at most 15000 mm³, at most 10000 mm³, or at most at most 8000 mm³.

The inventive 3D tissue culture may or may not comprise a vascular network in all embodiments of the invention, in particular, the inventive 3D tissue culture may comprise only cells of a single differentiation lineage, e.g. neural cells or organ, such as neural, gastric, connective, cartilage, bone, bone marrow, cardiac, kidney, vascular, breast or ductal-lobular, retinal, prostate, intestinal, gastric, lung, endothelium or liver tissue. This outcome may be controlled by the use of suitable differentiation factors as disclosed above. It is also possible to allow some variation in differentiation but maintain stringent differentiation to tissues of only one germ layer selected from mesoderm, endoderm, and ectoderm. Furthermore, the 3D tissue culture may be homogenously constituted from said cell of one differentiation lineage. Accordingly, other differentiation lineages may not be present, such as a connective tissue layer on the 3D tissue culture.

The 3D tissue culture may express certain differentiation expression markers, or lack expression of such expression markers as signals of a specific differentiation.

Preferably, said tissue culture comprises neural tissue and wherein the cancerous tissue is a neural tissue tumor.

Preferably the 3D tissue culture comprises cells, which express DLX2. DLX2 is expressed in cells of ventral forebrain identity. Preferably this tissue type is comprised in the inventive tissue.

Preferably the 3D tissue culture comprises cells, which express GSX2. GSX2 is expressed in cells of dorsal-lateral ganglionic eminence and caudal ganglionic eminence identity. Preferably this tissue type is comprised in the inventive tissue.

Preferably the 3D tissue culture comprises cells, which express NKX2-1. NKX2-1 is expressed in cells of ventral-medial ganglionic eminence identity. Preferably this tissue type is comprised in the inventive tissue.

Preferably the 3D tissue culture comprises cells, which express LHX6. LHX6 is expressed in cells of a subregion of ventral-medial ganglionic eminence identity. Preferably this tissue type is comprised in the inventive tissue.

Preferably the 3D tissue culture comprises cells, which express FoxG1. FoxG1 is expressed in cells of dorsal cortex identity. Preferably this tissue type is comprised in the inventive tissue.

Preferably the 3D tissue culture comprises cells, which express TBR1. TBR1 is expressed in cells of dorsal forebrain identity. Preferably this tissue type is comprised in the inventive tissue.

Preferably the 3D tissue culture comprises cells, which express TBR2. TBR2 is expressed in cells of dorsal cortical identity. Preferably this tissue type is comprised in the inventive tissue.

The inventive 3D tissue culture may contain a region of invasion or fusion between the tissue (part) of non-cancerous cells and the tissue (part) of cancerous cells. Such a region of invasion or fusion may allow the cells of the tissue types to be juxtaposed, resulting in a tissue that is a continuous tissue where one side is of the cancerous type, while the other side is of the non-cancerous type.

Preferably, non-cancerous tissue is at least at the core of the tissue and the cancerous tissue at least at the surface of the tissue. Since the aggregates are usually not disrupted before cultivation in the 3D matrix and the aggregate continues growing to the state of the 3D tissue culture in the 3D biocompatible matrix, which enhances growth and differentiation to in vivo-like lineages, and give that carcinogenesis is preferably on surface contact of the aggregate or the 3D tissue culture, by consequence, the first carcinogenic mutations will happen in cells on the surface. Other aggregate or tissue treatment to introduce carcinogenesis may be injection, accordingly, the cancer growth may start at the place of injection, which may be the core of the 3D tissue culture. “At least” means that the cells are found in the specified tissue location but may also be found in other parts of the tissue. In case of non-invasive cancerous cells, the cancerous cells may remain at their original location, such as the surface of the tissue. In case of invasive cells, the cancerous cells may be found throughout the tissue. For example, MYC-OE neoplastic cells are usually non-invasive. In case of GBM neoplastic cells, the cancerous cells grow not only on the surface, but also invade into the core of the tissue. Normal non-cancerous cells also grow on the surface of organoids.

Also provided is a method of testing or screening a candidate compound or agent for carcinogenesis or for its effect on cancer tissue, comprising contacting cells or a tissue in a method of the invention with the candidate compound or agent or contacting a tissue of the invention with the candidate compound or agent and maintaining said contacted tissue in culture, and observing any changes in the tissue as compared to said tissue without contacting by said candidate compound. Likewise, the invention provides exposing the tissue or the cells in the inventive method to a condition instead of contacting it with a candidate compound. Such a condition may be e.g. elevated temperature, limited nutrients or altered redox potential, to which cancer cells may react and exhibit a different behaviour or growth rate as compared to behaviour or growth without exposure to said condition. Accordingly, the inventive 3D tissue culture and the method of its generation can also be used as a research tool to study the effects of any chemical (compounds, e.g. drugs or other stimuli), (biological) agents (e.g. a virus, like an oncolytic virus and/or a Flavivirus) environmental (e.g. temperature, pressure, light exposure, redox potential, nutrients, irradiation) influences on growth and activity of cells in the tissue, in particular of the cells undergoing carcinogenesis. Temperature changes are preferably elevated temperature; altered nutrients are e.g. lowered glucose or other carbohydrate energy sources, increased fat or fatty acids; altered redox potential may e.g. be the addition of oxidizing agents or reducing agents or antioxidants, like vitamin C; light may be UV light; irradiation may be by alpha or beta radiation sources; a virus may be an oncolytic virus or a Flavivirus, a retrovirus or a DNA virus. In an inventive tissue that also comprises non-cancerous cells, it is further possible to compare the effects on the cancer cells to the effects on the non-cancerous cells of the same or a different 3D tissue culture. Accordingly, it is possible to identify cancer specific compounds, agents or environmental factors that have a stronger effect on cancer cells than non-cancer cells. In this case, compounds, agents or environmental factors may be eligible cancer therapy candidates, vs. compounds or agents or environmental factors that kill cancerous and non-cancerous cells indiscriminately. The cancer specific effect preferably kills or growth-inhibits 2 or more cancer cells for every non-cancerous cell. Preferably, this ratio is 3 or more, 4 or more, 5 or more, 10 or more, 20 or more or 100 or more cancer cells for every non-cancer cell. A therapeutic candidate thus classified may be subject for further caner tests, e.g. in an animal model or in patients.

The candidate compound or agent may be analysed and selected according to a desired property on the development of cancer in the 3D tissue culture. For example, compounds or agents may be analysed for their potential to slow or even halt cancer growth. Also, it is possible to screen for their ability to destroy tumor or cancer cells. Such effects can be screened in comparison to the non-cancerous cells, which are preferably less affected by such detrimental effects than the cancer cells, if the candidate compound should be further considered as a cancer treatment drug. Any kind of activity of the inventive cells or tissue, including metabolic turn-over or signalling can be searched for in a candidate compound or agent. In essence, the inventive highly differentiated tissue can be used as a model for tissue behaviour testing on any effects of any compound. Such a method might also be used to test therapeutic drugs, intended for treating cancer, for having side-effects on non-cancerous cells as can be observed in the inventive tissue culture. As said, instead of testing or screening a candidate compound or agent, also environmental conditions can be analysed for the same effects and purposes. Such effects may be elevated temperatures, such as 40° C. and above, or reduced nutrients like withdrawal of a carbohydrate or mineral source.

A candidate drug as candidate compound or agent may be a biomolecule, like a protein, peptide, nucleic acid, or comprise or be composed of such biomolecules, such as a virus, or a small molecule inhibitor. Small molecules are usually small organic compounds having a size of 5000 Dalton or less, e.g. 2500 Dalton or less, or even 1000 Dalton or less. The candidate drug, agent or compound may be known for other indication and/or a known chemical compound. Such known compounds are e.g. disclosed in compound databases such as www.selleckchem.com, which collects inhibitor compound information, including the cellular target of a compound. Preferably, the candidate compound is an inhibitor of an oncogene, in particular an oncogene that has been artificially introduced according to the inventive methods, either targeted or by random mutagenesis (and that was then identified) according to the inventive methods described above. Many and any compound can be screened, for example for target gene EGFR, www.selleckchem.com lists more than 50 inhibitors that are all eligible screening targets, of course. Further candidate compounds are virus particle, in particular infectious virus particles, including wild type viruses or attenuated viruses. Effective viruses are called oncolytic viruses due to their anti-cancerous effect, although lysis of a tumor or of cancer cells is not strictly necessary. Such a virus that was found to be a viable treatment option for cancer is the Zika Flavivirus. The examples herein show its oncolytic potential in a neural tumor organoid. Its use is a further aspect of the invention.

In particular preferred embodiments, the candidate compound or agent is tested or screened for its effects on any cancerous or proliferative central nervous system disorder, in particular preferred glioblastoma, neuroblastoma or CNS-PNET (central nervous system primitive neuro-ectodermal tumor). Accordingly, the inventive tissue comprises such a disorder or in the inventive method such a disorder is created in the carcinogenesis step. Such a method is particularly used to screen for or test potential therapeutic compounds or agents.

The inventive 3D tissue culture can also be implanted into an animal. Possible implantation sites are anywhere in the animal, such as subcutaneous or in an organ cavity, such as near the kidney. Said screening or testing methods can also be performed on in the animal model, e.g. by administering a candidate compound or agent to the animal. Example animals are e.g. non-human primates, rodents, non-human mammals, etc. The candidate agent or drug may be administered in combination with a pharmaceutical preparation. Example pharmaceutical preparations are further described below. The invention thus also provides animals comprising the inventive 3D tissue culture.

In a further aspect, the invention provides Zika virus for use as a oncolytic virus. In particular provided is Zika virus for use in the treatment of nervous system cancer. Related thereto is a method of treating a nervous system cancer in a patient comprising treating a patient having nervous system cancer with Zika virus to remove said cancer. Further provided is the use of Zika virus in the manufacture of a medicament for the treatment of nervous system cancer. Also provided is a method of treating nervous system cancer cells with Zika virus. The cancer cells may be in a patient or in vitro, e.g. in a 3D tissue culture as described. Nervous system cancer may e.g. be a brain cancer or a spinal cord cancer. As shown herein, Zika virus (ZIKV) preferentially infects tumor cells in organoids and severely impairs tumor growth. The results show that cerebral organoids can be used to test strategies for brain tumor therapy and to shows the use of ZIKA as an oncolytic virus.

Oncolytic viruses and their use to treat cancer are well-known in the art (see WO 1998/035028 A2, WO 2001/053506 A2, WO 2002/067861 A2, WO 2004/078206 A1, WO 2017/070110 A1, WO 2017/085175 A1, WO 2017/132552 A1, WO 2017/120670 A1; Russell et al. Nat Biotechnol. 30(7): 658-670 (2012)) for use alone (i.e. as only anti-cancer drug) or in combination with other cancer therapies, in particular chemotherapy (WO 2008/043576 A1, WO 2017/121925 A1). The inventive therapy using Zika virus is an oncolytic virus therapy and particular aspects can be used as known in the art, such as administering the virus in a formulation suitable for viral delivery and/or stability. Candidate oncolytic viruses can be tested or screened for oncolytic effects in the inventive methods and tissue cultures as candidate compounds.

Zika virus (ZIKV) is a mosquito-borne flavivirus distributed throughout much of Africa and Asia. Infection with the virus may cause acute febrile illness that clinically resembles dengue fever. It has been characterized and is available in the art (Haddow et al., PLoS neglected tropical diseases 6(2) 2012:e1477, incorporated herein by reference). Any strain can be used, such as the African strain or Asian strain, including any of its lineages, including MR 766, ArD 41519, IbH 30656, EC Yap, P6-740 or FSS13025. In addition, Zika virus may be attenuated or recombinantly engineered to include further antigens or attenuation modifications. The genome of the Zika virus of the invention preferably still has at least 85% or at least 90% or at least 95% sequence identity to any one of lineages MR 766, ArD 41519, IbH 30656, EC Yap, P6-740 or FSS13025 as deposited and reviewed by Haddow et al., 2012, supra.

In some embodiments, the invention provides a method of treating cancer in a subject in need thereof, comprising administering an oncolytic Zika virus described herein or compositions thereof to the subject. In some embodiments, the subject is a mouse, a rat, a rabbit, a cat, a dog, a horse, a non-human primate, or a human. In some embodiments, the oncolytic virus or compositions thereof are administered intravenously, subcutaneously, intratumorally, intramuscularly, intranasally, parenterally, or intraperitoneally. The virus can be administered systemically or topically. In case of metastasizing tumors, systemic administration is preferred. In case of singular tumor, a topical administration to the tumor site or cancerous organ is also possible.

The invention further provides a pharmaceutical composition comprising a replication competent Zika virus and a stabilizer or carrier for said virus. The pharmaceutical composition may be used in the treatment of neural cancer cells. A stabilizer may be any stabilizer for viral formulations, preferably to create a shelf-life of at least 3 months, at room temperature or under cooled storage, such as at 1° C. to 8° C. An example stabilizer may be a carbohydrate (U.S. Pat. No. 8,142,795 B2), including disaccharides (U.S. Pat. No. 6,231,860 B1) or serum proteins like albumin (U.S. Pat. No. 6,210,683 B1) or salts comprising Mg²⁺and Ca²⁺ions (U.S. Pat. No. 3,915,794 A) or glutamic acid and arginine (U.S. Pat. No. 4,337,242 A) or combinations thereof. Of course, the concentration of the stabilizer shall be adapted to achieve a stabilized effect, such as maintaining the infectious virus in solution, for at least 14 days or more like 2-3 months or more. The composition may further comprise a sensitizer such as to remove or reduce protection of the cancer by the patient's immune system. An example sensitizer is an IFN inhibitor (Russell et al., 2012, supra) or a check-point inhibitor (WO 2017/120670 A1 ). The Zika virus may be a wild type virus, which may require isolation of the patient in order to prevent infection of bystanders, or the Zika virus may be life-attenuated to mitigate infection capacity and contagion of bystanders. Because Zika virus only causes a mild infection in besides having cancer otherwise healthy persons with the exception of pregnant women, Zika virus may be wild type. Accordingly, the person treated with Zika virus is not a pregnant female in such a case.

The pharmaceutical composition may comprise a carrier. As used herein, “carrier” includes any and all solvents, dispersion media, vehicles, coatings, diluents, isotonic and absorption delaying agents, buffers, carrier solutions, suspensions, dispersants, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. In one embodiment, a composition comprising a carrier is suitable for parenteral administration, e.g., intravascular (intravenous or intraarterial), intraperitoneal or intramuscular administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with a viral vector or nucleic acid molecule, use thereof in the pharmaceutical compositions of the invention is contemplated.

The pharmaceutical forms suitable for injectable use include sterile aqueous solutions or dispersions and sterile powders for the extemporaneous preparation of sterile injectable solutions or dispersions (U.S. Pat. No. 5,466,468, incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The composition may further comprise antibacterial and/or antifungal agents or other preservatives to increase shelf-life. Of course, sterile or the presence shall not prevent Zika virus' oncolytic capability. Sterility therefore does not extend to the removal or inactivation of Zika virus. Likewise, the preservative shall not preserve against Zika virus.

The composition may further comprise an antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

An example composition may comprise combinations of all of these components, such as the Zika virus, a stabilizer for the virus, preferably PEG, a carrier, and an antioxidant, preferably further a sensitizer. The composition may be sterile with the exception of the presence of Zika virus, which shall remain infectious, and/or comprise a preservative that is not harmful to Zika virus.

The patient to be treated may have been diagnosed with neuronal cancer or neural cancer, such as neuroblastoma or glioblastoma. For example, the patient may have a glioma (glial cell tumor), e.g. Gliomatosis cerebri, Oligoastrocytoma, Choroid plexus papilloma, Ependymoma, Astrocytoma (Pilocytic astrocytoma, Glioblastoma multiforme), Dysembryoplastic neuroepithelial tumor, Oligodendroglioma, Medulloblastoma, Primitive neuroectodermal tumor; a neuroepitheliomatous tumor, e.g. Ganglioneuroma, Neuroblastoma, Atypical teratoid rhabdoid tumor, Retinoblastoma, Esthesioneuroblastoma; or a nerve sheath tumor, e.g. Neurofibroma (Neurofibrosarcoma, Neurofibromatosis), Schwannoma, Neurinoma, Acoustic neuroma, Neuroma. Any such tumor may also be modelled by the inventive 3D tissue culture or organoid. Also, the inventive method of treating neural cancer cell may comprise diagnosing or detecting neural cancer cells and then treating the cells with Zika virus according to the invention. The treatment with Zika virus may take precautionary preparations such as isolating the patient to prevent further infection in other subjects, in particular humans.

The invention also relates to a kit for providing a tissue culture according to the invention. The kit may comprise (i) a transfection vector comprising an oncogene transgene or a construct for disruption of a tumor suppressor, (ii) a 3D biocompatible matrix, preferably further comprising (iii) a tissue differentiation agent, a stem cell culturing medium, a nucleofection medium or a combination thereof. The kit can be used in the inventive method. Preferably the kit comprises any further compound or means as disclosed above for the inventive method. In particular, preferred, the kit also comprises a marker as disclosed above in order to label mutated cells. The marker is preferably an expression marker, such as a fluorescent protein. The 3D matrix has been described in length above—preferably it comprises a collagenous hydrogel or any other embodiment disclosed herein. The kit further preferably comprises a differentiation agent, a stem cell culturing medium, a nucleofection medium or a combination thereof. Such media are known in the art and usually include one or more of the following components:

Differentiation agent: any one of the differentiation factors as disclosed above, preferably a neuronal differentiation factor, these are suitable for creating neuronal 3D tissue cultures; Stem cell culturing medium: N2 supplement, B27 supplement, insulin, 2-mercaptoethanol, glutamine, non-essential amino acids, or any combination thereof (see WO 2014/090993 A1);
Nucleofection medium: e.g. Dulbecco's modified Eagle medium (DMEM) or other nutrient and mineral source, glutamine, and FCS or other serum or serum replacement (see also U.S. Pat. No. 7,732,175 B2); DMEM or the other nutrient source is preferable in a range of 80-95 w.-% of the medium. FCS or other serum or serum replacement is preferably 5-20 w.-% of the medium. The nucleofector medium should be suitable for nucleofection, preferably for electroporation.

In addition to these components, the kit may also comprise suitable containers, such as flasks or vials to hold its components, preferably separately for each component or medium.

The invention is further illustrated in the following figures and examples, without being limited to these embodiments of the invention.

### FIGURES

FIG. 1. Nucleofection of genome-editing constructs into neural stem/precursor cells (NS/PCs) of cerebral organoids. a, Schematic of the culture system of cerebral organoid system and nucleofection strategy. Example images of each stage are presented. EBs were electroporated at the end of neural induction stage, right before the matrigel embedding to initiate tumorigenesis. EB, embryoid body; bFGF, basic fibroblast growth factor; hESCs, human embryonic stem cells; hiPSCs, human induced pluripotent stem cells; RA, retinoic acid. b, Immunofluorescence photographs revealed that nucleofected cells (GFP, green) in EBs at the end of neural induction stage are NS/PCs (SOX1, red; N-CAD, red; NES, red; arrowheads), but neither mesodermal cells (BRA, red; FOXF1, red; arrows) nor endodermal cells (SOX17, red; CD31, red; arrows). N-CAD: N-CADHERIN; NES: NESTIN; BRA: BRACHYURY. Scale bar: b, upper panel: 200 μm; lower panel: 100 μm.

FIG. 2. Clonal mutagenesis in organoids induces tumorous overgrowth. Immunofluorescence photographs (a) and quantification of the GFP fluorescence intensity of organoids 1 day (b) and 1 month (c) after nucleofection. Result showed that EBs from all groups contains similar amount of nucleofected cells 1 day after nucleofection, while organoids from four groups, including MYC, CDKN2A⁻/CDKN2B⁻/EGFR^{OE}/EGFRvIII^{OE}, NF1⁻/PTEN⁻/p53⁻, EGFRvIII/PTEN⁻/CDK2A⁻, exhibit dramatic overgrowth of GFP⁺cells in cerebral organoids 1 month after nucleofection. Scale bar: a: 1 day: 200 μm; 1 month: 500 μm.

FIG. 3. MYC^{OE} and GBM-like neoplastic cerebral organoids have distinct transcriptional profiles and cellular identities. a, Principle component analysis (PCA) of the top 500 variable genes between normal cells from CTRL organoids and tumor cells from different neoplastic cerebral organoid groups. b, Venn diagrams showed overlap of genes differentially expressed (DESeq, adjusted p value<0.05) between the Cluster 2 (MYC^{OE}, n=3) and the Cluster 3 (GBM-1, GBM-2, GBM-3, n=7) relative to the CTRL organoids (Cluster 1; n=3). The p value for overlaps were calculated by hypergeometric test. c, KEGG pathway enrichment analysis revealed the differences in signalling pathways between neoplastic cerebral organoids from the Cluster 2 and the Cluster 3. d, The heatmap shows normalised expression levels for differentially expressed genes (adjusted absolute log2fc value>1 or <−1 and adjusted p value<0.05) between Cluster 2 and Cluster 3 (n=3 for Cluster 2 and n=7 for Cluster 3 from one experiment) selected from differentially expressed genes between human primary CNS-PNET and GBM tumors. The heatmap was created from log2 (Transcripts Per Kilobase Million, TPM) transformed data that was row (gene) normalised using the “Median Center Genes/Rows” and “Normalise Genes/Rows” functions to report data as relative expression between samples. e, Low-magnification images of DAPI (blue) and GFP (green) staining of control and neo-plasm groups 4 months after nucleofection. f-k, Representative immunofluorescence images and quantification of four-month-old organoids from CTRL, MYC^{OE}, and GBM-1. The staining was performed from six independent experiments with similar results. Quantification was performed on organoids from three independent experiments. Statistical analysis was performed using one-way ANOVA with Dunnett's test. Data are presented as mean±SD, with details of sample sizes and values, as well as adjusted p value in Source Data. *, p<0.05; **, p<0.01; ***, p<0.001. Scale bar: c: 100 μm.

FIG. 4. Neoplastic organoids expanded upon renal subscapular xenografts. a, Schematic of renal capsule xenograft procedure. Two-month-old neoplastic organoids were implanted into kidney capsule of nude mice, and tissues were collected 1.5 months after. b, Brightfield and immunofluorescence photographs showed that neoplastic organoids were expanded, while control organoids were largely absorbed. c, Photograph of H&E staining of neoplastic organoids in renal capsule. Glial cells are pointed by arrows, and neurons are pointed by arrowhead. d, Immunohistochemical photographs of glial marker GFAP, precursor marker SOX1, and cell cycle marker Ki67 on implanted organoids. e, Photographs of H&E staining of implanted MYC^{OE} organoids showed that MYC^{OE} neoplasm exhibit CNS-PNET-like histopathological features. f, Immunohistochemical photographs of neuronal marker MAP2 revealed that MYC^{OE} neoplasm barely differentiated into neurons. Scale bar: b: 500 mm; c, d, f: 200 μm and 50 μm (inset); e: 1000 μm; e′, e″, e′″: 50 μm.

FIG. 5. GBM neoplastic cerebral organoids exhibit features of GBM invasion. a-c, Representative images of the tumor-normal interface in GBM-1 neoplastic cerebral organoids. Images are representative of at least three independent experiments. d, Immunohistochemical staining of GFAP in GBM-like neoplastic cerebral organoids. Images are representative of two independent renal implantations. Dotted black lines indicate the boundary between implanted neoplastic cerebral organoids and murine kidney. Dotted red line indicates the renal tubule. Arrowheads indicate invaded tumor cells. e, Hierarchical clustering analysis of GBM invasiveness-relevant genes from four-month-old organoids (n=3 for CTRL organoids; n=4 for MYC^{OE}, n=4 for GBM-1, n=4 for GBM-2, and n=3 for GBM-3 neoplastic cerebral organoids, from three independent cultures for each group). The heatmap was created from log2 (TPM) transformed data that was row (gene) normalised using the “Median Center Genes/Rows” and “Normalise Genes/Rows” functions to report data as relative expression between samples. f, Representative immunofluorescence staining of neoplastic cerebral organoids from GBM-1 group for the indicated mesenchymal marker and invasiveness markers; GFP is also shown. Images are representative of two independent experiments. Scale bar: a, 1000 mm; b and c, 200 mm; d, 25 μm; f: 100 μm.

FIG. 6. Using brain neoplastic organoid model to investigate potential brain tumor therapies. a, b, Images (a) and quantification of FACS sorting (b) assay revealed that EGFR inhibitors Afatinib was able to diminish most of GFP⁺tumor cells in GBM-1 (n=6) and GBM-3 (n=3) neoplastic cerebral organoids, but exhibited no effect on tumor cells in MYC^{OE} and GBM-2 neoplastic cerebral organoids compared to DMSO treatment. The percentage of GFP⁺cells in total cells from the drug-treated groups were normalized to the percentage of GFP⁺cells from DMSO-treated neoplastic cerebral organoids. Statistical analysis of quantification was performed using unpaired two-tailed Student's t-test. c, Schematic of ZIKV infection and experimental setups. d, Immunofluorescence images of GFP and ZIKV of CTRL organoids treated with either MOCK or ZIKV, as well as ZIKV-treated MYC^{OE} and GBM-1 neoplastic cerebral organoids. e, Quantification of ZIKV infection ratio showed significantly higher infection ratio of GFP⁺tumor cells from all neoplastic cerebral organoid groups compared to non-tumor cells from CTRL organoids or neoplastic cerebral organoids. Statistical analysis of quantification was performed using one-way ANOVA with Dunnett's test. f, Immunofluorescence images of neural precursor marker MUSASHI1 (MSI1) of CTRL organoids treated with either MOCK or ZIKV, as well as ZIKV-treated MYC^{OE} and GBM-1 neoplastic cerebral organoids. g, Immunofluorescence images of apoptosis marker activated Caspase3 (CASP3) of CTRL organoids treated with either MOCK or ZIKV, as well as ZIKV-treated MYC^{OE} and GBM-1 neoplastic cerebral organoids. h, Quantification of percentage of CASP3⁺apoptosis cells showed that ZIKV-infection induced significantly more cells apoptosis in tumor regions compared to MOCK-treated tumor regions, and MOCK- or ZIKV-treated non-tumor regions. CTRL-ZIKV Statistical analysis of quantification was performed using one-way ANOVA with Dunnett's test. i, Quantification of the yields of progeny ZIKV by analysing the percentage of ZIKV-infected Vero cells exposed to the supernatant from CTRL and neoplastic cerebral organoids at 4 dpi. Compared to supernatant from CTRL organoids, significantly more Vero cells were infected exposed to the supernatant from neoplastic cerebral organoid groups. Statistical analysis of quantifications was performed using one-way ANOVA with Dunnett's test. j, Epifluorescence iamges of neoplastic cerebral organoids from MYC^{OE} groups upon MOCK or ZIKV exposure at 0, 6, and 14 dpi. k, FACS sorting analysis of GFP⁺tumor cell proportion in different neoplastic cerebral organoid groups upon MOCK treatment at 14 dpi. The percentage of GFP⁺cells in total cells from the ZIKV-treated groups were normalized to the percentage of GFP⁺cells from MOCK-treated neoplastic cerebral organoids. Statistical analysis of quantification was performed using unpaired two-tailed Student's t-test. *, p<0.05; **, p<0.01; ***, p<0.001. Scale bar: a, d, f, g, j, 1000 μm.

FIG. 7. The strategy to introduce gene aberrations into neural stem/precursor cells in cerebral organoids. a, Schematic of the strategy of genome-editing techniques to introduce oncogene amplification and/or tumor suppressor mutation/deletion. Sleeping Beauty transposon system was used to integrate oncogene-expression and GFP-expression elements into genome. CRISPR-Cassystem was applied to introduce mutation/deletion of tumor suppressors. b, Quantification of cellular identities of nucleofected cells in EBs 1 day after nucleofection by immunofluorescence staining on serial cryo-sections. Results showed that 100% of GFP⁺cells are SOX1⁺(n=402), N-CADHERIN⁺(N-CAD) (n=451), and NESTIN⁺(NES) (n=433) neural stem/precursor cells. None of GFP⁺cells is BRACHYURY⁺(BRA) (n=398) or FOXF1⁺(n=356) mesodermal cells, or SOX17⁺(n=328) or CD31⁺(n=267) endodermal cells. c, d, Immunofluorescence images (c) and quantification (d) of adherent cell culture of dissociated EBs 1 day after nucleofection. Results showed that 100% of GFP⁺cells are SOX1⁺(n=549), N-CADHERIN⁺(N-CAD) (n=403), and NESTIN⁺(NES) (n=461) neural stem/precursor cells. None of GFP⁺cells is BRACHYURY⁺(BRA) (n=474) or FOXF1⁺(n=402) mesodermal cells, or SOX17⁺(n=334) or CD31⁺(n=415) endodermal cells. Scale bar: c, 50 μm.

FIG. 8. Verification of gene aberrations introduced by genome-editing techniques. a, RNA-seq and RT-PCR analysis showed that tumor cells from MYC^{OE} neoplastic cerebral organoids exhibit high MYC expression levels. b, Three example sequences of CRISPR-Cas9 targeting CDKN2A and CDKN2B locus in tumor cells from GBM-1 neoplastic cerebral organoids. RNA-seq and RT-PCR analysis showed that tumor cells from GBM-1 neoplastic cerebral organoids exhibit high expression levels of both EGFR and EG-FRvIII. c, Three example sequences of CRISPR-Cas9 targeting NF1, PTEN, and TP53 locus in tumor cells from GBM-2 neoplastic cerebral organoids. d, Three example sequences of CRISPR-Cas9 targeting CDKN2A and PTEN locus in tumor cells from GBM-3 neoplastic cerebral organoids. RNA-seq and RT-PCR analysis showed that tumor cells from GBM-3 neoplastic cerebral organoids exhibit high expression level of EGFRvIII, but not EGFR.

FIG. 9. Low-magnification images revealed that 4-month-old neoplastic organoids showed brain tumor subtype-specific cellular identities. a, Immunofluorescence photographs of control and neoplastic groups 1 day and 4 months after nucleofection confirmed the tumor-initiation capability of genetic disruptions. b, Immunofluorescence photographs of DAPI (blue) and GFP (green) staining of control and neoplastic groups 4 months after nucleofection. c-h, Immunofluorescence photographs and quantification of neuronal marker HuC/D (c), precursor marker SOX2 (d, red), cell cycle marker Ki67 (e, red), CNS-PNET marker CD99 (f, red), as well as glial marker S100β (h, red) and GFAP (g, red). Scale bar: a: upper panel: 200 μm, lower panel: 1000 μm; b-g: 1000 μm.

FIG. 10. High-magnification images revealed that 1-monthold neoplastic organoids showed brain tumor subtype-specific cellular identities. a, Immunofluorescence photographs of control and neoplastic groups 1 day and 1 months after nucleofection confirmed the tumor-initiation capability of genetic disruptions. b, Immunofluorescence photographs of DAPI (blue) and GFP (green) of control and tumor groups 1 month after nucleofection. c-e, Immunofluorescence photographs and quantification of neuronal marker HuC/D (c, red), precursor marker SOX2 (c, cyan), cell cycle marker Ki67 (d, red), as well as glial marker S100β (e, red). Scale bar: a: upper panel: 200 μm, lower panel: 1000 μm; b, 1000 μm; c-h: 100 μm.

FIG. 11. Low-magnification images revealed that one-month-old Neoplastic organoids showed brain tumor subtype-specific cellular identities. a, Immunofluorescence photographs of control and neoplastic groups 1 day and 1 month after nucleofection confirmed the tumor-initiation capability of genetic disruptions. b, Immunofluorescence photographs of DAPI (blue) and GFP (green) staining of control and neoplastic groups 1 month after nucleofection. c-e, Immunofluorescence photographs and quantification of neuronal marker HuC/D (c, red), precursor marker SOX2 (c, cyan), cell cycle marker Ki67 (d), as well as glial marker S100β (e). Scale bar: a: upper panel: 200 μm, lower panel: 1000 μm; b-h: 1000 μm.

FIG. 12. In vivo expansion of neoplastic cerebral organoids. Neoplastic cerebral organoids from MYC^{OE} group and GBM-1 group were implanted into kidney capsule. Engrafted kidneys were analysed at 1 week and 1.5 months after xenograft to evaluate the in vivo expansion of neoplastic cerebral organoids.

FIG. 13. Drug testing assay showed the drug screening potential of neoplastic organoids. a, Schematic of luciferase assay-based drug testing strategy on neoplastic organoids. b, Quantification of relative luciferase activity revealed that EGFR inhibitors Afatinib and Erlotinib significantly reduced luciferase activity in GBM1 (CDKN2A⁻/CDKN2B⁻/EGFR^{OE}/EGFRvIII^{OE}) neoplastic organoids (CTRL group: n=3; DMSO: n=9; Canertinib: n=9; Pelitinib: n=8; Afatinib: n=9; Gefitinib: n=10; Erlotinib: n=9). Normalized luciferase activity was presented. Statistical analysis of quantifications was performed using one-way ANOVA with Dunnett's test. **, p<0.01.

FIG. 14. ZIKV exhibits various tropism toward different subtypes of neural cells. a,b, Immunofluorescence images and quantifications of triple-staining of GFP (green), ZIKV (magenta), and different neural cell subtype markers, including neural precursor marker SOX2 (cyan) and MSI1 (cyan), glial marker S100β (cyan), and neuronal marker HuC/D (cyan), as well as a double staining for ZIKV (magenta) and GFP (green) that represent tumor cells. Results showed significantly more GFP+ tumor cells co-localized with ZIKV staining compared to other non-tumor neural cell types. In addition, ZIKV infection ratios of SOX2⁺and MSI1⁺non-tumor precursor cells are significantly higher than HuC/D⁺non-tumor neurons, which match the previous observations. c,d, Immunofluorescence images and quantifications of ZIKV infection ratio of different cell types within tumors from different neoplastic cerebral organoid groups. Results demonstrated that cell type tropism within tumor regions. Statistical analysis of quantifications was performed using one-way ANOVA with Dunnett's test. *, p<0.05; ***, p<0.001. Scale bar: a, c, 100 μm.

FIG. 15. Neoplastic cerebral organoids produce more ZIKV progeny. a, Immunofluorescence images of ZIKV-infected Vero cells exposed to the supernatant from CTRL and neoplastic cerebral organoids at 4 dpi. Cells were stained by DAPI (blue), and ZIKV was stained as green. b, qPCR analysis revealed the significantly higher ZIKV gene expression in neoplastic cerebral organoids compared to CTRL organoids upon ZIKV infection. The ZIKV vRNA level of neoplastic cerebral organoids were normalized to the ZIKV vRNA level of CTRL organoids. Statistical analysis of quantifications was performed using one-way ANOVA with Dunnett's test. ***, p<0.001. Scale bar: 100 μm.

FIG. 16. ZIKV infection in tumor regions of neoplastic cerebral organoids resulted in a remarkable more cell apoptosis. a, Immunofluorescence images of cell apoptosis marker CASP3 (red) of ZIKV-infected non-tumor and tumor regions, as well as MOCK-treated non-tumor and tumor regions. DAPI (blue) was stained for nuclei, and GFP (green) was staining to represent tumor cells. b, Immunofluorescence images of ZIKV staining and cell apoptosis marker CASP3 of MYC^{OE} neoplastic cerebral organoids upon MOCK-treatment, and ZIKV-infection at 6 dpi and 14 dpi. Scale bar: a, 100 μm; b, 1000 μm.

### EXAMPLES

### Example 1. Materials and Methods

### 1.1 Plasmid Constructs and Materials

For overexpression constructs, based on the Sleeping Beauty Transposase System, the CMV promoter from pCMV(CAT)T7-SB100 (Addgene cat. No.: 34879; Mátés et al., 2009, Nat Genet, 41, 753-61) was replaced with CAG promotor from pCAGEN (Addgene cat. No.: 11160; Matsuda and Cepko, 2004, Proc. Natl. Acad. Sci. U.S.A., 101, 16-22). IRDR-R and IRDR-L sequences from pT2/LTR7-GFP (Addgene cat. No.: 62541; Wang et al., 2014, Nature 516, 405-9) were cloned into pCAGEN to produce pCAG-GS/IR. cDNAs used for overexpression were amplified from human cDNA and cloned into the MCS of pCAG-GS/IR. With the help of sleeping beauty transposase SB100X (pCAG-SB100X), CAG-GFP and CAG-oncogenes were integrated into the genome of cells in organoids. To introduce gene mutations, short guide RNAs of tumor suppressors were cloned into CRISPR/Cas9 vector pX330-U6-Chimeric_BB-CBh-hSpCas9 (Addgene cat. No.: 42230; Ran et al., 2013, Nat Protoc, 8, 2281-308). All cloning primers are listed in the Tables 1.

TABLE 1
Primers for cloning oncogenes into sleeping
beauty construct

| Gene | | |
|---|---|---|
| symbols | Primers | |
| MYC | up- | GACGGCGCGCCCGCCACCATGCTGGATTTTTTTC |
| | stream | GGGTAG (SEQ ID NO: 1) |
| | down- | GACACCGGTTTACGCACAAGAGTTCCGTAG |
| | stream | (SEQ ID NO: 2) |
| EGFR/EG | up- | GACGGCGCGCCCGCCACCATGCGACCCTCCGGGA |
| FRvIII | stream | CG (SEQ ID NO: 3) |
| | down- | GACACCGGTTCATGCTCCAATAAATTCACTG |
| | stream | (SEQ ID NO: 4) |
| PDGFRA | up- | GACGGCGCGCCCGCCACCATGGGGACTTCCCATC |
| | stream | CGGCGTTC (SEQ ID NO: 5) |
| | down- | GACACCGGTTTACAGGAAGCTGTCTTCCACCAG |
| | stream | (SEQ ID NO: 6) |
| CDK4 | up- | GACGGCGCGCCCGCCACCATGGCTACCTCTCGA |
| | stream | TATGAGC (SEQ ID NO: 7) |
| | down- | GACACCGGTTCACTCCGGATTACCTTCATCC |
| | stream | (SEQ ID NO: 8) |
| MDM2-B | up- | GACGGCGCGCCCGCCACCATGTGCAATACCAACA |
| | stream | TGTCTG (SEQ ID NO: 9) |
| | down- | GACACCGGTCTAGGGGAAATAAGTTAGCAC |
| | stream | (SEQ ID NO: 10) |
| H3F3A- | up- | CATTTTGGCAAAGAATTCCCTCGATACCGGGGG |
| K27M/ | stream | CGCGCCCGCCACCATGGCTCGTACAAAGCAGAC |
| H3F3A- | | TGC (SEQ ID NO: 11) |
| G34R | down- | CGGGAATGCTAGCAATCATTGGTTGATCAGCTT |
| | stream | TGTTACCGGTTTAAGCACGTTCTCCACGTATG |
| | | (SEQ ID NO: 12) |

1.2 Human Embryonic Stem Cell (hESC) And Human Induced Pluripotent Stem Cell (iPSC) Culture

Feeder-free (FF) H9 hESCs were obtained from WiCell with verified normal karyotype and contamination free. FF H9 hESCs were cultured in a feeder-free manner on Matrigel (Corning, hESC-qualified Matrix)-coated plate with mTeSR medium (Stemcell Technologies). Feeder-dependent (FD) H9 hESCs were obtained from WiCell with verified contamination-free. FD H9 hESCs were cultured on CF-1-gamma-irradiated mouse embryonic stem cells (MEFs) (GSC-6001G, Global Stem) according to WiCell protocols. All cell lines were routinely checked for mycoplasma-negative. All stem cells were maintained in a 5% CO₂ incubator at 37° C. Standard procedures were used for culturing and splitting hESCs as explained previously (Lancaster et al., 2013, Nature, 501, 373-9). All hESCs were authenticated using Infinium PsychArray-24 Kit (Illumina).

### 1.3 Generation of Cerebral Organoids

Cerebral organoids were cultured as previous described (Lancaster et al., 2013 Nature 501, 373-379; WO 2014/090993 A1; both incorporated herein by reference). Briefly, to make EBs (embryoid bodies), hESCs/hiPSCs were trypsinized into single cells, and 9,000 cells were plated into each well of an ultraplow-binding 96-well plate (Corning) in human ES medium containing low concentration basic fibroblast growth factor (bFGF, 4 ng/ml) and 50 μM Rho-associated protein kinase (ROCK) inhibitor (Calbiochem). EBs were fed every three days for 6 days then transferred to neural induction media to form neuroepithelial tissues. After 5-7 days in neural induction media, EBs were embedded into droplets of Matrigel (Corning) and cultured in differentiation medium without vitamin A (Diff-A). Finally, the EB droplets were transferred to 10 cm-dish containing differentiation medium with vitamin A (Diff+A) and cultured on an orbital shaker. Media were changed weekly.

### 1.4 Nucleofection of Organoids to Induce Gene Mutation/Amplification

In order to initiate the brain tumors, we introduced the tumor suppressor mutations and/or oncogene amplifications on neuroepithelial cells at the end of neural induction culture, right before the Matrigel embedding. Briefly, 10-15 EBs were collected, resuspended in nucleofetion reagent (Nucleofector™ kits for human stem cells, Lonza) containing plasmids and transferred into nucleofection vials. Nucleofection was performed according to the manufacturer's protocol. After electroporation, EBs were carefully transferred to 6 cm-dish containing neural induction medium, and cultured at 37° C. incubator for 4 hours. Then nucleofected EBs were embedded into Matrigel and cultured for organoids as described. The neoplastic cerebral organoids with significant overgrowth of GFP⁺cels were selected for further investigations, in which the samples were randomly allocated.

### 1.5 Adherent Cell Culture of Dissociated EBs.

One day after nucleofection, the EBs were trypsinised at 37° C. for 20 min to make single cell suspension. Then cells were plated on the poly-D-lysine- and laminin-coated coverglasses in neural induction medium with ROCK inhibitor, and cultured in a 5% CO₂ incubator at 37° C. The further immunofluorescence staining and analysis were performed the day after.

### 1.6 RNA-Seq Analysis

Organoids from control and neoplastic groups were collected 40 days and four months after nucleofection, and trypsinised with shaking at 37° C. for half an hour. GFP⁺cells were sorted according to the example gating strategy, and total RNA was isolated using RNeasy Micro kit (Qiagen) according to the manufacturer's instruction. RNA concentration and quality were analysed using RNA 6000 Nano Chip (Agilent Technologies). Messenger RNA (mRNA) was enriched using SMART-Seq v4 Ultra Low Input RNA Kit (TaKaRa) according to manufacturer's protocol. Libraries were prepared using NEB Next Ultra Directional RNA library Prep kit for Illumina (NEB). Barcoded samples were multiplexed and sequenced 50 bp SE on a HighSeq 2500 (Illumina). mRNA sample isolation, library preparation, and sequencing were performed at the VBCF NGS Unit (www.vbcf.ac.at).

The unstranded reads were screened for ribosomal RNA by aligning with BWA (v0.7.12) against known rRNA sequences (RefSeq). The rRNA subtracted reads were aligned with TopHat (v2.1.1) against the Homo sapiens genome (hg38). Microexonsearch was enabled. Additionally, a gene model was provided as GTF (UCSC, 2015_01, hg38). rRNA loci are masked on the genome for downstream analysis. Aligned reads are subjected to Transcripts Per Kilobase Million (TPM) estimation with Kallisto (v0.43.0). Furthermore, the aligned reads were counted with HTSeq (v0.6.1; intersection-nonempty) and the genes were subjected to differential expression analysis with DESeq2 (v1.12.4).

Before the bioinformatics analysis, the expression of oncogenes according to the genome editing manipulation was checked, and one four-month-old sample from GBM-3 neoplastic cerebral organoid group was excluded from the further analysis because of the failure of introducing the overexpression of EGFRvIII.

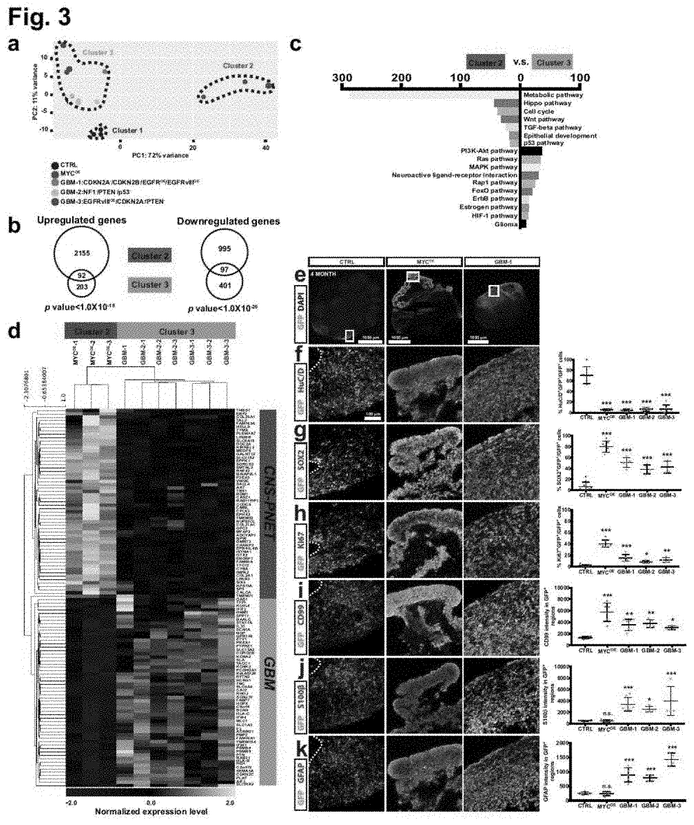

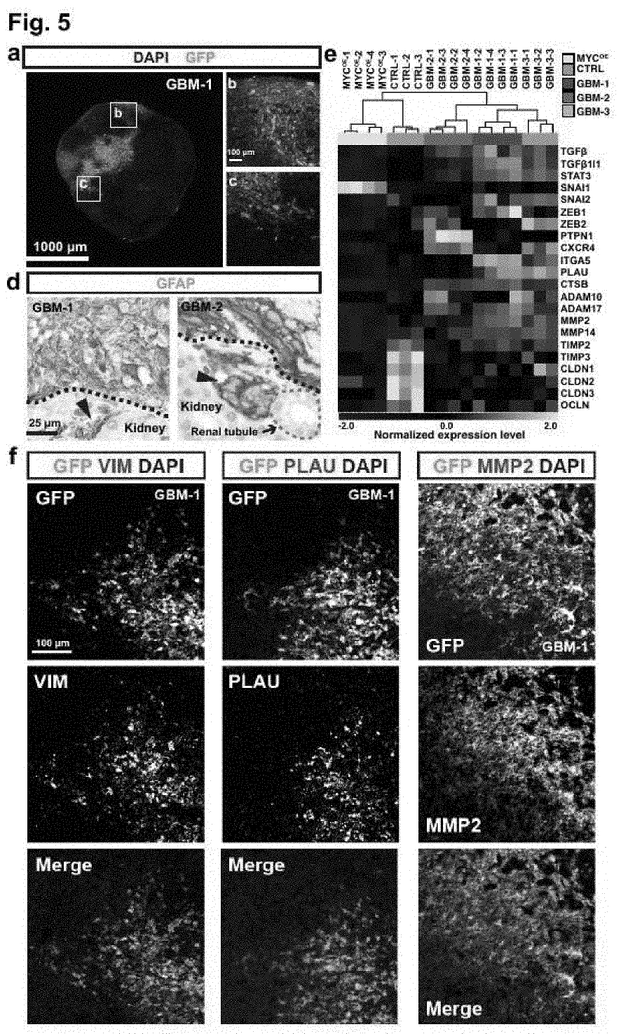

PCA was performed using the top 500 variable genes between normal cells from CTRL organoids and tumor cells from different neoplastic cerebral organoid groups. Venn diagram hypergeometric test was performed on differentially expressed genes between Cluster 2 or Cluster 3 versus CTRL, and KEGG pathway enrichment analysis were performed on differentially expressed genes between Cluster 2 and Cluster 3 with an adjusted absolute log2fc value>0.5 and adjusted p value<0.05. Venn diagram hypergeometric test was performed via R language. KEGG pathway enrichment was analysed using DAVID Bioinformatics (david.ncifcrf.gov) (Huang et al, 2009, Nature Protocol, 4, 44-57). The heatmap of RNA-seq was generated using MeV (Saeed et al., 2003, BioTechniques 34, 374-8). For the heatmap of tumor-subtype gene profiling (FIG. 3c), the differentially expressed genes between Cluster 2 and Cluster 3 (adjusted absolute log2fc value>1 or <−1 and adjusted p value<0.05) were selected from the differentially expressed gene list (adjusted absolute log2fc value>1 or <−1 and adjusted p value<0.05) from human primary tumor transcriptome analysis (Sturm et al., 2016, Cell, 164, 1060-1072). For the heatmap of hierarchical clustering analysis of GBM invasiveness-relevant genes (FIG. 5e), differential expressed genes from any individual GBM groups versus CTRL organoids with an adjusted absolute log2fc value>0.5 and adjusted p value<0.05 were selected. The heatmap was created from log2 (TPM) transformed data that was row (gene) normalised using the “Median Center Genes/Rows” and “Normalise Genes/Rows” functions to report data as relative expression between samples.

### 1.7 Verification of Genome Alteration Introduced by SB and CRISPR/Cas9

To test whether the genome editing techniques actually altered the genome in tumor cells, GFP⁺tumor cells were FACS sorted for genomic DNAs isolation for genotyping and for RNAs to verify the expression of oncogenes. RNAs were isolated using RNeasy Micro kit (Qiagen), and cDNA was synthesised according to previous description (Bagley et al., 2017, Nature Methods, 14, 743-751). RT-PCRs for. MYC, EGFR, EGFRvIII, and TBP were performed using the primers listed in Table 2. Genomic DNAs were isolated using DNeasy Blood & Tissue Kits (Qiagen) according to the manufacturer's instruction. The CRISPR/Cas9 targeted genome locus of tumor suppressor genes were amplified using primers listed in the Table 3. The PCR products were inserted into T vector (Promega) according to the manufacturer's instruction. Nighty-six colonies per gene were cultured for sequencing.

TABLE 2
Primers for RT-PCR

| Gene | | |
|---|---|---|
| symbols | Primers | |
| MYC | Top | TCGGATTCTCTGCTCTCCTC (SEQ ID |
| | | NO: 33) |
| | Bottom | CCTGCCTCTTTTCCACAGAA (SEQ ID |
| | | NO: 34) |
| EGFR/ | Top | CGGGCTCTGGAGGAAAAG (SEQ ID |
| EGFRvIII | | NO: 35) |
| | Bottom | GCCCTTCGCACTTCTTACAC (SEQ ID |
| | | NO: 36) |
| TBP | Top | GGGCACCACTCCACTGTATC (SEQ ID |
| | | NO: 37) |
| | Bottom | CGAAGTGCAATGGTCTTTAGG (SEQ ID |
| | | NO: 38) |
| ZIKV | Top | TTGGTCATGATACTGCTGATTGC (SEQ ID |
| | | NO: 39) |
| | Bottom | CCTTCCACAAAGTCCCTATTGC (SEQ ID |
| | | NO: 40) |

TABLE 3
Primers for cloning tumor suppressor guide
RNAs into CRISPR-Cas9 construct

| Gene | | |
|---|---|---|
| symbols | Primers | |
| CDKN2A | Top | CACCGTCCCGGGCAGCGTCGTGCAC (SEQ ID |
| | | NO: 13) |
| | Bottom | AAACGTGCACGACGCTGCCCGGGAC (SEQ ID |
| | | NO: 14) |
| CDKN2B | Top | CACCGACGGAGTCAACCGTTTCGGG (SEQ ID |
| | | NO: 15) |
| | Bottom | AAACCCCGAAACGGTTGACTCCGTC (SEQ ID |
| | | NO: 16) |
| NF1 | Top | CACCGCTCGTCGAAGCGGCTGACCA (SEQ ID |
| | | NO: 17) |
| | Bottom | AAACTGGTCAGCCGCTTCGACGAGC (SEQ ID |
| | | NO: 18) |
| PTEN | Top | CACCGAACTTGTCTTCCCGTCGTGT (SEQ ID |
| | | NO: 19) |
| | Bottom | AAACACACGACGGGAAGACAAGTTC (SEQ ID |
| | | NO: 20) |
| p53 | Top | CACCGTCGACGCTAGGATCTGACTG (SEQ ID |
| | | NO: 21) |
| | Bottom | AAACCAGTCAGATCCTAGCGTCGAC (SEQ ID |
| | | NO: 22) |
| RB1 | Top | CACCGCGGTGGCGGCCGTTTTTCGG (SEQ ID |
| | | NO: 23) |
| | Bottom | AAACCCGAAAAACGGCCGCCACCGC (SEQ ID |
| | | NO: 24) |
| ATRX | Top | CACCGAAATTCCGAGTTTCGAGCGA (SEQ ID |
| | | NO: 25) |
| | Bottom | AAACTCGCTCGAAACTCGGAATTTC (SEQ ID |
| | | NO: 26) |
| SMARCB1 | Top | CACCGAGAACCTCGGAACATACGG (SEQ ID |
| | | NO: 27) |
| | Bottom | AAACCCGTATGTTCCGAGGTTCTC (SEQ ID |
| | | NO: 28) |
| PTCH1 | Top | CACCGCAGATAGTCCCGGTCCGGCG (SEQ ID |
| | | NO: 29) |
| | Bottom | AAACCGCCGGACCGGGACTATCTGC (SEQ ID |
| | | NO: 30) |
| CTNNB1 | Top | CACCGAAACAGCTCGTTGTACCGCT (SEQ ID |
| | | NO: 31) |
| | Bottom | AAACAGCGGTACAACGAGCTGTTTC (SEQ ID |
| | | NO: 32) |

### 1.8 Renal Subcapsular Engrafting

All procedures were performed in accordance with institutional animal care guidelines. Briefly, adult MF1 nu/nu male mice (8 to 12 weeks) were anesthetized with ketamine solution. After disinfecting the surgical site with 70% alcohol, a 1.5-2 cm incision was made and the kidney was carefully exteriorized. The renal capsule was incised for 2-4 mm using a pipette tip, and a capsule pocket for the grafts was made using a blunted glass Pasteur pipette. Two-month-old organoids from each group were carefully implanted under the renal capsule, respectively. Then kidney was gently replaced back into the retroperitoneal cavity. During the exteriorization, the kidney was kept hydration by applying PBS with penicillin/streptomycin. The kidneys were collected one and half months after xenograft for further analysis.

### 1.9 Immunofluorescence and Immunohistochemistry

For immunofluorescence staining, tissues were fixed in 4% paraformaldehyde (PFA) at 4° C. for overnight. The tissues were dehydrated in 30% sucrose overnight, embedded in Tissue-Tek (VWR), and then cryosectioned at 16 μm. For immunofluorescence staining, sections were blocked and permeabilized in 0.25% Triton X-100 and 4% normal donkey serum (NDS) in PBS at room temperature (RT). Sections were incubated at 4° C. with primary antibody in 0.1% Triton-X-100 and 4% NDS in PBS. After washing three times for 10 min with PBS, sections were incubated with secondary antibodies in 0.1% Triton-X-100 and 4% NDS in PBS and DAPI consecutively for visualizing the immunostains. The primary and secondary antibodies were used for immunofluorescence were listed in Tables 4, 5. Images were captured using a confocal microscope (Zeiss LSM 780). Quantification of images from three independent preparations of neoplastic organoids was performed using Fiji.

TABLE 4
Primary Antibodies

| | | | Catalog | | Appli- |
|---|---|---|---|---|---|
| Antigen | Species | Company | No. | Dilution | cation |
| BRACHYURY | Goat | R&D Systems | AF2085 | 1:200 | IF |
| CD31 | Mouse | Dako | M0832 | 1:200 | IF |
| CD99 | Rabbit | Abcam | ab108297 | 1:500 | IF |
| Cleaved | Rabbit | Cell Signaling | 9661S | 1:200 | IF |
| Caspase-3 | | Technology | | | |
| Flavivirus | Mouse | Merck Millipore | MAB10216 | 1:600 | IF |
| antigen | | | | | |
| FOXF1 | Goat | R&D Systems | AF4798 | 1:200 | IF |
| GFAP | Rabbit | DAKO | Z0334 | 1:500 | IF&IHC |
| GFP | Chicken | Abcam | ab13970 | 1:500 | IF&IHC |
| HuC/D | Mouse | Abcam | ab21271 | 1:100 | IF |
| Ki67 | Mouse | BD Pharmingen | 550609 | 1:100 | IF&IHC |
| MAP2 | Rabbit | Merck Millipore | MAB3418 | 1:500 | IHC |
| MSI1 | Rabbit | Abcam | ab21628 | 1:200 | IF |
| N-CADHERIN | Mouse | BD Biosciences | 610920 | 1:500 | IF |
| NESTIN | Mouse | BD Transduction | 611658 | 1:200 | IF |
| | | Laboratories | | | |
| S100β | Rabbit | Abcam | ab52642 | 1:200 | IF |
| SOX1 | Goat | R&D Systems | AF3389 | 1:200 | IF&IHC |
| SOX2 | Rabbit | Abcam | ab97959 | 1:1000 | IF |
| SOX17 | Goat | R&D Systems | AF1924 | 1:100 | IF |
| Zika | Rabbit | GeneTex | GTX133314 | 1:600 | IF |

TABLE 5
Secondary Antibodies

| | Recog- | Fluoro- | | Catalog | Dilu- | Appli- |
|---|---|---|---|---|---|---|
| Host | nizes | phore | Company | No. | tion | cation |
| Donkey | Chicken | Alexa | Jackson | 703- | 1: 500 | IF |
| | | Fluor | Immuno | 605-155 | | |
| | | 488 | | | | |
| Donkey | Rabbit | Alexa | Invitrogen | A10042 | 1: 500 | IF |
| | | Fluor | | | | |
| | | 568 | | | | |
| Donkey | Rabbit | Alexa | Invitrogen | A31573 | 1: 500 | IF |
| | | Fluor | | | | |
| | | 647 | | | | |
| Donkey | Mouse | Alexa | Invitrogen | A31571 | 1: 500 | IF |
| | | Fluor | | | | |
| | | 647 | | | | |
| Donkey | Mouse | Alexa | Invitrogen | A10036 | 1: 500 | IF |
| | | Fluor | | | | |
| | | 568 | | | | |
| Donkey | Goat | Alexa | Invitrogen | A11057 | 1: 500 | IF |
| | | Fluor | | | | |
| | | 568 | | | | |
| Goat | Mouse | Alexa | Invitrogen | A21144 | 1: 500 | IF |
| | IgG2b | Fluor | | | | |
| | | 568 | | | | |
| Goat | Rabbit | n/a | Dako | E0432 | 1: 500 | IHC |
| Goat | Chicken | n/a | Abcam | Ab97135 | 1: 500 | IHC |
| Rabbit | Goat | n/a | Dako | F0250 | 1: 500 | IHC |

For histologic and immunohistochemical staining, tissues were fixed in 4% paraformaldehyde overnight. Fixed tissues were rinsed in PBS, dehydrated by immersion in an ascending ethanol gradient (70%, 90%, and 100% ethanol), embedded in paraffin, and sectioned at a thickness of 2 to 5 μm. Sections were stained by a routine Hematoxylin and Eosin (H&E) protocol in a Microm HMS 740 automated stainer. Immunohistochemistry was performed using the Leica Bond III automated immunostainer. The primary and secondary antibodies used in this study were listed in Table 4, 5. Slides were reviewed with a Zeiss Axioskop 2 MOT microscope and images were acquired with a SPOT Insight digital camera. Slides were also scanned with a Pannoramic 250 Flash II Scanner (3D Histech). Digital slides were reviewed and images acquired with the Pannoramic Viewer software (3D Histech). Slides were reviewed by a board certified Veterinary Comparative Pathologist (A.K.).

### 1.10 Drug Testing on Neoplastic Organoids

For drug testing, neoplastic organoids were first grown for 2 months, followed by drug treatment for 40 days. EGFR inhibitors Afatinib (www.selleckchem.com, cat. No.: S1011), Erlotinib (www.selleckchem.com, cat. No.: S7786), Gefitinib (www.selleckchem.com, cat. No.: S1025), Canertinib (www.selleckchem.com, cat. No.: S1019), and Pelitinib (Sigma-Aldrich, cat. #: 257933-82-7) (final concentration 1 μM) were applied, and DMSO was used as control. After drug treatment, neoplastic organoids were trypsinized for single cell preparation, followed by FACS sorting analysis. Total cell numbers were counted to evaluate the cytotoxicity of the drugs.

### 1.11 ZIKV Stock Production and Infections

The ZIKV strain (H/PF/2013) was passaged in Vero cells to establish a viral stock. Briefly, Vero cells (maintained in DMEM medium supplemented with 10% Fetal Bovine Serum, and 2 mM L-Glutamine) were infected with ZIKV at MOI 0.1 and incubated at 37° C., in 5% CO₂ humidified atmosphere. At 3 days post infection, cell supernatants from infected cells were harvested and purified by centrifugation at 1500 rpm for 10 min to remove cellular debris. Supernatant of non-infected cells was used as MOCK. Supernatants were aliquoted and stored at −80° C. To determine viral titer, confluent Vero cells in 96-well plates were infected with serially diluted ZIKV stock. The assay was carried out in eight parallels wells for each dilution with the last column of 96-well plate as cell control without virus. The cells were incubated at 37° C. in 5% CO₂ humidified atmosphere. At 5 days post infection, the appearance of cytopathic effects (CPE) were examined by microscope. The TCID₅₀ was calculated from the CPE induced in the cell culture. All ZIKV experiments were conducted under Biosafety Level 2 Plus (BSL2+) containment. For infections of organoids, 130 to 160-day-old CTRL or neoplastic organoids cultured in Diff+A medium were transferred into 6 or 10 cm dishes. ZIKV stock and equivalent volume of MOCK were diluted in Diff+A medium to 0.5×10\hat{ }6 TCID₅₀ particles/ml and 2 ml/organoid of diluted stocks (for a total of 10\hat{ }6 TCID₅₀ particles/organoid) were added to the dish and incubated at 37° C., in 5% CO₂ humidified atmosphere on an orbital shaker. Media were changed every 4 days. All the experiments performed in ZIKV studies were done for at least three times independently.

### 1.12 Statistical Analysis

Statistical analyses were performed with GraphPad Prism 7. Statistical analysis of quantifications performed was done using unpaired Student's two-tailed t-test for significance between two experimental groups in all experiments, except for those involving NGS-based approaches. Statistically significant threshold was accepted as p<0.05.

### Example 2. Clonal Mutagenesis in Organoids Induces Tumorous Overgrowth

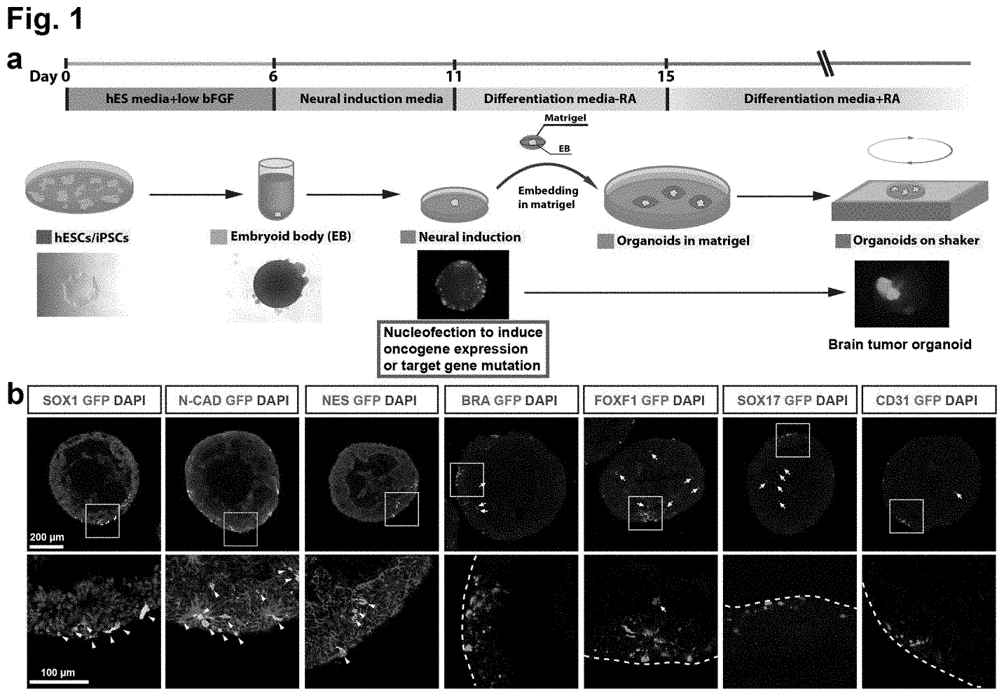

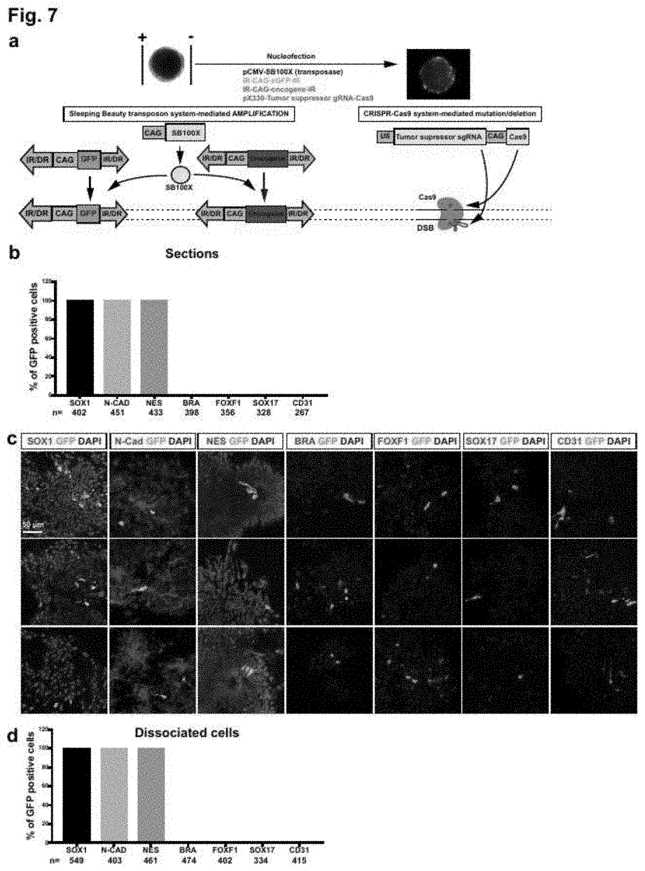

Brain tumors are characterized by a wide variety of DNA aberrations that either cause oncogene overexpression or loss of tumor suppressor gene function (McLendon et al., 2008, Nature, 455, 1061-8). Importantly, a recent re-classification of brain cancer subtypes includes DNA aberrations as a defining feature (Louis et al., 2016, Acta Neuropathol, 131, 803-20), highlighting the need for genetically defined human brain cancer models. To recapitulate a wide variety of tumorigenic events, we combined Sleeping Beauty (SB) transposon-mediated gene insertion with CRISPR/Cas9-based mutagenesis. Combinations of plasmids encoding (1) the SB transposase, (2) GFP flanked by SB inverted repeats (IRs), (3) any oncogene flanked by IRs and (4) multiple plasmids expressing the Cas9 nuclease together with one or many guide RNAs (gRNAs) were introduced into cerebral organoids by electroporation before matrigel embedding (FIG. 7). At this stage of the protocol (FIG. 1a), neural induction is complete and neural stem and progenitor cells (NS/PCs) are expanding on the surface of embryoid bodies (EBs). Immunostaining of EBs 24 h after nucleofection of pCAG-GFP showed that 100% of GFP⁺cells are SOX2⁺, N-CADHERIN⁺(N-CAD⁺), and NESTIN⁺(NES⁺) NS/PCs (FIG. 1b and FIG. 7b-d). None of GFP⁺cells are BRACHYURY⁺(BRA+) or FOXF1⁺mesodermal cells, or SOX17⁺or CD31⁺endodermal cells (FIG. 1b and FIG. 7b-d). Thus, the electroporated plasmids are exclusively delivered into NS/PCs, which are often presumed to be cells of origin for brain cancers (Chen et al., 2012, Cell, 149, 36-47).

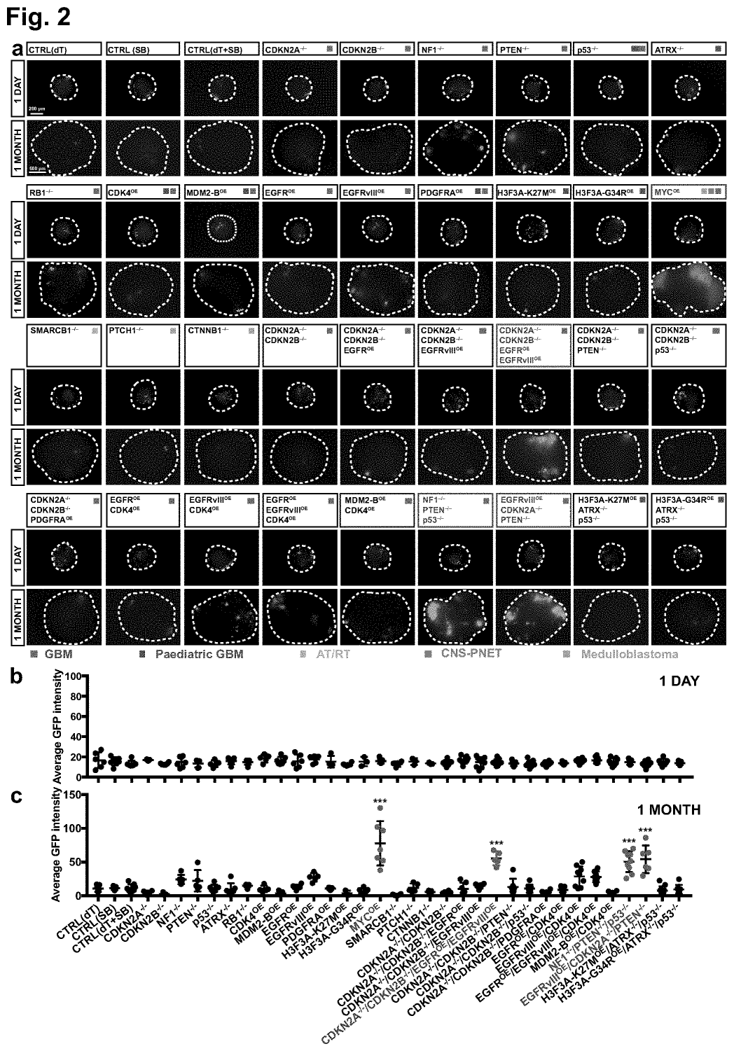

To ask whether tumor-like overgrowth can be induced in cerebral organoids, we tested 18 single gene mutations or amplifications as well as 15 of the most common clinically-relevant combinations observed in GBM (McLendon et al., 2008, Nature, 455, 1061-8) (Table 6). As most electroporated cells carry the CAG-GFP insertion, GFP intensity was used to quantify proliferation of cells carrying gene aberrations. One day after electroporation, EBs from all groups contained similar amounts of GFP⁺cells (FIG. 2a, b). One month later, however, striking overgrowth of GFP⁺cells was observed in organoids carrying the MYC-amplification (MYC^{OE}), and in organoids carrying CDKN2A⁻/CDKN2B⁻/EGFR^{OE}/EGFRvIII^{OE}, NF1⁻/PTEN⁻/p53, and EGFRvIII^{OE}/CDKN2A⁻/PTEN⁻(FIG. 2a, c). As these combinations of gene aberrations are commonly found in GBM, we refer to them as GBM-1, GBM-2, and GBM-3, respectively. Thus, cerebral organoids can be used to test the tumorigenic capacity of different gene aberrations induced within the same cell of origin.

TABLE 6
Genetic aberrations

| Groups with gene aberrations | Tumor subtypes |
|---|---|
| CDKN2A | GBM |
| CDKN2B | GBM |
| NF1 | GBM |
| PTEN | GBM |
| p53 | GBM, Pediatric GBM |
| ATRX | Pediatric GBM |
| RB1 | GBM |
| CDK4 | GBM, Pediatric GBM |
| MDM2-B | GBM, Pediatric GBM |
| EGFR | GBM |
| EGFRvIII | GBM |
| PDGFRA | GBM, Pediatric GBM |
| H3F3A-K27M | Pediatric GBM |
| H3F3A-G34R | Pediatric GBM |
| MYC | GBM, CNS-PNET, MB |
| SMARB1 | AT/RT |
| PTCH1 | MB |
| CTNNB1 | MB |
| CDKN2A/CDKN2B | GBM |
| CDKN2A/CDKN2B/EGFR | GBM |
| CDKN2A/CDKN2B/EGFRvIII | GBM |
| CDKN2A/CDKN2B/EGFR/EGFRvIII | GBM |
| CDKN2A/CDKN2B/PTEN | GBM |
| CDKN2A/CDKN2B/p53 | GBM |
| CDKN2A/CDKN2B/PDGFRA | GBM |
| EGFR/CDK4 | GBM |
| EGFRvIII/CDK4 | GBM |
| EGFR/EGFRvIII/CDK4 | GBM |
| MDM2-B/CDK4 | GBM |
| NF1/PTEN/p53 | GBM |
| EGFRvIII/CDKN2A/PTEN | GBM |
| H3F3A-K27M/ARTX/p53 | Pediatric GBM |
| H3F3A-G34R/ARTX/p53 | Pediatric GBM |

Abbreviation
GBM: glioblastoma;
CNS-PNET: center nervous system primitive neuroectodermal tumor;
MB: medulloblastoma
AT/RT: atypical teratoid/rhabdoid tumor

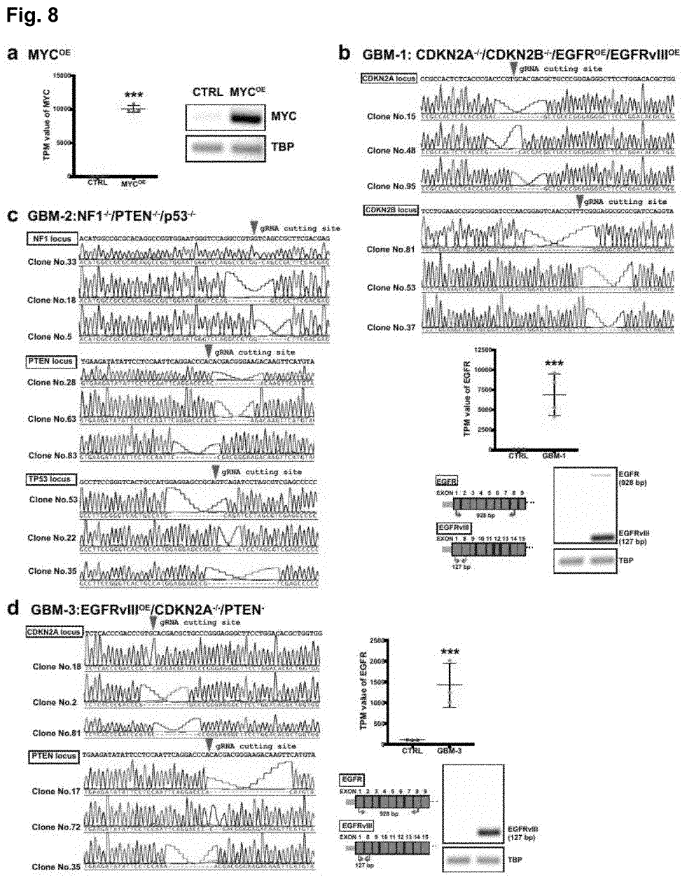

To confirm that the genome editing techniques actually altered the genome in tumor cells, the expression of oncogenes and/or sequences of CRISPR-targeting regions were analysed, and the results confirmed that tumor cells from different groups carried the expected gene mutations/amplifications (FIG. 8a-d). Thus, cerebral organoids can be used as a platform to test the tumorigenic capacity of different gene aberrations induced within the same cell of origin.

### Example 3. MYC^{OE} and GBM-Like Tumors have Distinct Transcriptional Profiles

To test whether brain tumor-like organoids resemble distinct brain tumor-subtypes, we performed transcriptome analysis on GFP⁺cells isolated by FACS. Principal component analysis (PCA) of genes expressed differently between groups identified three distinct clusters. Cluster one included all control (CTRL) organoids which harbour only CAG-GFP and a control gRNA targeting tdTomato (FIG. 3a). Cluster two included the organoids carrying the MYC^{OE} construct, while cluster three contained the organoids carrying genetic aberrations found in GBM (GBM-1, GBM-2, GBM-3). Importantly, the majority of genes deregulated in the MYC^{OE} group are distinct from those deregulated in the GBM-groups (FIG. 3b), confirming the PCA analysis. KEGG pathway analysis via the DAVID Bioinformatics tools (Huang et al., 2009, Nature Protocol, 4, 44-57) confirmed a glioma signature in organoids in Cluster 3 and showed upregulation of the PI3K-Akt, Rap1, ErbB, HIF1, NF-kappa B, and Estrogen signaling pathways that are also relevant for GBM (Gutmann et al., 1997, Oncogene, 15, 1611-6; Clark et al., 2012, NEO, 14, 420-IN13; Mayer et al., 2012, Int. J. Oncol., 41, 1260-70; Puliyappadamba et al., 2014, Mol Cell Oncol, 1, e963478) (FIG. 3c). In the organoids from the Cluster 2, we detected upregulation of metabolic pathways and cell cycle genes, but also the Hippo, WNT, TGFβ, and p53 signalling pathways that are known to be connected to MYC (Rogers et al., 2012, British Journal of Cancer, 107, 1144-52; Hutter et al., 2017, Genes, 8, 107-19; Atkins et al., 2016, Curr. Biol., 26, 2101-13) (FIG. 3c). In addition, the MYC^{OE} group showed upregulation of an epithelial development signature, suggesting a CNS-PNET-like neoplasm, which originates from neuroepithelial cells.

To confirm the similarity of the organoid tumors with primary tumor tissues, we tested the genes differentially expressed between CNS-PNET and GBM (Sturm et al., 2016, Cell 164, 1060-72) for their expression in neoplastic organoids. Hierarchical clustering revealed that neoplastic organoids from the MYC^{OE} group showed a strong CNS-PNET signature. Organoids from Cluster 3 exhibited upregulation of GBM genes (FIG. 3d) and we refer to this cluster as the GBM-group below. Taken together, our observations suggest that we succeeded in creating neoplastic organoids that recapitulate two subtypes of brain tumors by inducing distinct genetic modifications in the same cell of origin.

### Example 4. MYC^{OE} and GBM Organoid Tumors have Different Cellular Identities

To characterize the cellular identities of MYC^{OE} and GBM neoplastic organoids, we tested specific CNS-PNET and GBM markers 4 months after nucleofection. CNS-PNETs are characterized by undifferentiated SOX2⁺cells and high CD99 expression (Rocchi et al., 2010, J. Clin. Invest., 120, 668-80), while the glial markers S100β and GFAP and the proliferation marker Ki67 are diagnostic for GBM.

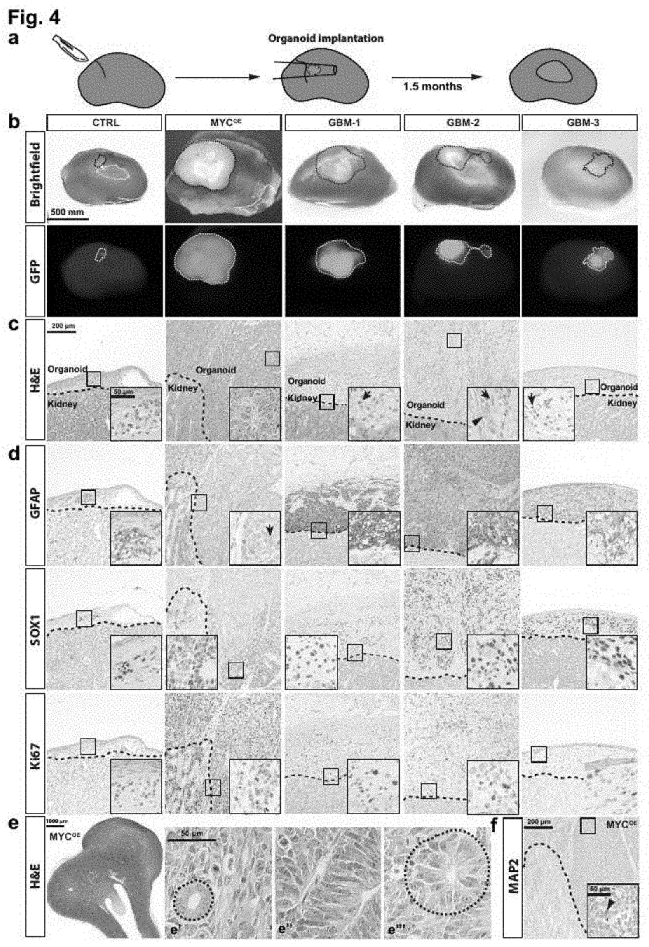

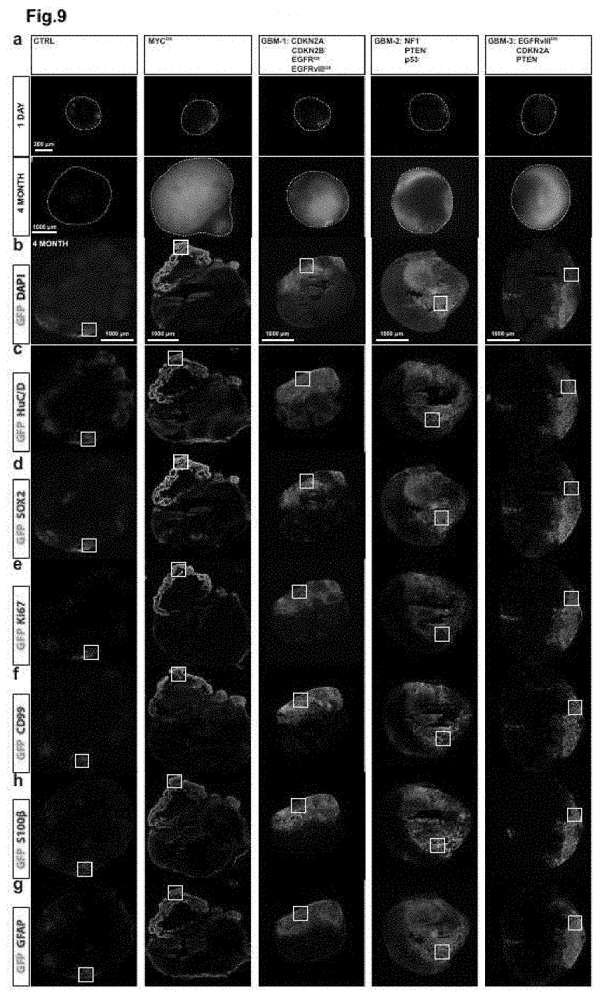

In CTRL organoids, the majority of GFP⁺cells were HuC/D⁺neurons (FIG. 3f and FIG. 9c), while only a small portion of GFP⁺cells maintained SOX2 (FIG. 3g and FIG. 9d) and Ki67⁺(FIG. 3h and FIG. 9e) and the glial markers S100β (FIG. 3j and FIG. 9h) and GFAP (FIG. 3k and FIG. 9g) were essentially absent in GFP⁺cells. In the MYC^{OE} group, very few GFP⁺cells are HuC/D⁺(FIG. 3f and FIG. 9c), or express the glial markers S100β (FIG. 3j and FIG. 9h) or GFAP (FIG. 3k and FIG. 9g). Instead, the most GFP⁺cells were SOX2⁺(FIG. 3g and FIG. 9d), and almost half of them expressed Ki67 (FIG. 3h and FIG. 9e). In addition, most MYC^{OE}/GFP⁺cells expressed high levels of CD99 antigen (FIG. 4i and FIG. 9f), further confirming their CNS-PNET-like cellular identities. In the GBM-relevant groups, GFP⁺regions were positive for S100β⁺(FIG. 3j and FIG. 9h) and GFAP⁺(FIG. 3k and FIG. 9g) glial cells and contained only few HuC/D⁺neurons (FIG. 3f and FIG. 9c). Compared with CTRL organoids, we also detected more SOX2⁺(FIG. 3g and FIG. 9d) and Ki67⁺(FIG. 3h and FIG. 9e) cells, which are also found in the central core of GBM tumors (Schmitz et al., 2007, British Journal of Cancer, 96, 1293-301). In addition, GFP⁺regions in GBM-relevant groups showed elevated CD99 levels (FIG. 4i and FIG. 9f), a feature also reported for GBM tissues (Seol et al., 2012, Genes & Cancer, 3, 535-49).

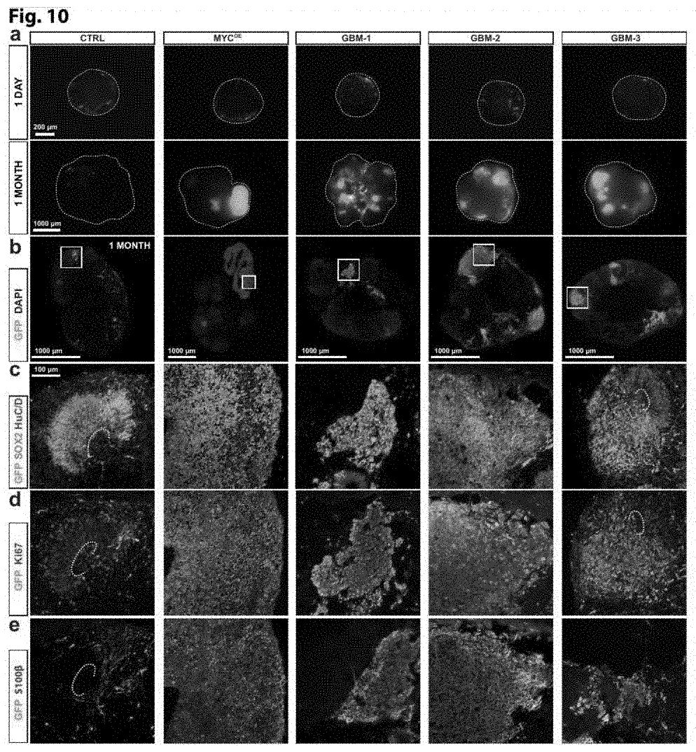

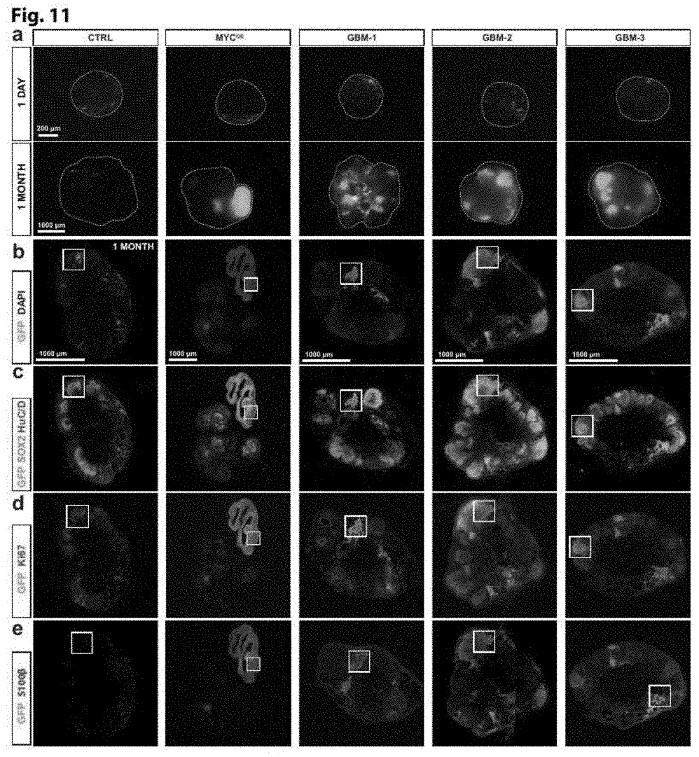

We also examined tissue organization in the various groups of organoid neoplasms. In CTRL organoids, GFP⁺cells located in the ventricular zone (labelled with dashed line) of rosette-like cortical regions, expressed SOX2 and Ki67, while GFP⁺/HuC/D⁺neurons were located in the basal cortical regions (FIG. 3e-k and FIG. 9b-g). In the MYC^{OE} group, GFP⁺cells formed both large sheets of cells and small rosette-like structures (FIG. 3e-k and FIG. 9b-g), which are also often observed in CNS-PNET tissues. GBM-groups, in contrast, showed a disorganized architecture with disruption of orderly cortical architecture (FIG. 3e-k and FIG. 9b-g). Noteworthy, staining of 1-month-old control organoids and neoplastic organoids showed similar trends of cellular identities and same histological features as 4-month-old organoids (FIG. 10a-e and FIG. 11a-e).

Thus, neoplastic organoids induced through generating distinct genetic aberrations recapitulate the establishment of cellular identities and histo-morphological structures of either CNS-PNET or GBM, starting from the same cell of origin.

### Example 5. Renal Subscapular Engrafting of Neoplastic Organoids

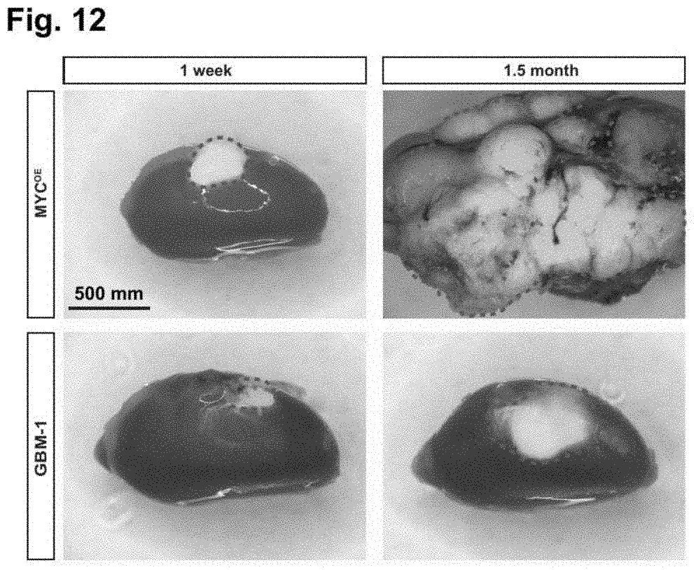

To confirm that organoid neoplasms can grow in vivo, we implanted them into renal subcapsular space of immunodeficient mice, an environment that can provide abundant blood supply to implanted cells (FIG. 4a). In controls, four out of five organoids were resorbed within six weeks and the remaining organoid was reduced to only a tiny cluster of cells (FIG. 4b) that had lost cellularity and architectural detail (FIG. 4c). Thirteen out of fifteen neoplastic organoids, in contrast, were retained and often expanded even beyond the renal capsule (FIG. 4b and FIG. 12). Transplanted organoids from the MYC^{OE} group proliferated massively often invading the adjacent renal cortex. They formed cell sheets and rosettes remarkably similar to CNS-PNET (FIG. 4c, e′, e″). Immunohistochemical analysis revealed many neuro-epithelial areas positive for the NS/PC marker SOX1 (FIG. 4d), but very few cells positive for the glial marker GFAP (FIG. 4d) or the neuronal marker MAP2 (FIG. 4f), indicating their primitive, poorly differentiated state. GBM groups instead displayed high expression of glial marker GFAP, NS/PC marker SOX1, and cell cycle marker Ki67 (FIG. 4d). GBM-1 and GBM-3 organoids displayed a glial (arrowhead) neoplasia like expansion (FIG. 4c), while GBM-2 showed glial (arrowhead) neoplasia like proliferation with additional cells of mature neuronal appearance (arrow) reminiscent of glioneuronal tumors (FIG. 4c). Thus, neoplastic organoids can engraft and expand in vivo and maintain their subtype identity upon renal transplantation into nude mice.

### Example 6: GBM-Like Neoplastic Cerebral Organoids are Suitable to Study Interaction Between Tumorous and Normal Tissues

Compared to other in vitro brain tumor models such as 2D cell cultures or 3D tumor spheres, a distinct feature of the inventive neoplastic cerebral organoids is that tumors were initiated by introducing gene aberrations in a very small portion of cells during cerebral organoid culture. This not only mimics human tumor initiation in vivo, but also results in a mixed structure which contains both tumor and normal tissues adjacent to each other. This advantage allowed this approach an ideal platform to study some essential tumor biological questions such as invasiveness, which is one of the main causes of high mortality in GBM patients.

GBMs are known to extensively infiltrate into adjacent brain parenchyma. During GBM progression, epithelial-mesenchymal transition (EMT) confers essential migratory and invasive capabilities to tumor cells. Therefore, high expression of transcription factors inducing EMT are observed in GBMs, which may also activate mesenchymal features in them. With respect to invasiveness, many proteases, including matrix metalloproteases, are also involved in the interaction between GBM tumor cells and the extracellular matrix (ECM).

To assess whether neoplastic cerebral organoids can be used to study the invasiveness of GBM, we evaluated the neoplastic and normo-cellular interface in GBM-like neoplastic cerebral organoids. We observed the invasive presence of GFP⁺tumor cells within normal regions (FIG. 5a-c). Small invasive foci of tumor cells that breached the renal capsule were also observed in the renal xenografts of GBM-group neoplastic cerebral organoids (FIG. 5d). To analyze the invasiveness of GBM-group tumor cells, RNA-seq analysis was further performed to compare the expression of invasion-related genes in tumor cells and normal cells from 4-month-old organoids. Hierarchical clustering analysis showed that, compared to CTRL organoids, the tumor cells from different GBM groups have higher expression level of GBM invasiveness genes, including EMT-related transcriptional factors (TGFβ, TGFβ1I1, STAT3, SNAI2, ZEB1, ZEB2), migration-related receptor (CXCR4), extracellular matrix molecules (ITGA5), proteases (PLAU, CTSB, ADAM10, ADAM17, MMP2, MMP14), respectively (FIG. 5e). In addition, tumor cells from GBM groups exhibit downregulation of many genes inhibiting tumor invasion compared to normal cells in CTRL organoids, such as tissue inhibitors of matrix metalloproteinases (TIMP2, TIMP3), and tight junction components (CLDN1, CLDN2, CLDN3, OCLN) (FIG. 5e). To confirm the RNA-seq results, immunostaining was performed using antibodies against the mesenchymal marker vimentin (VIM), invasion-associated proteases urokinase (PLAU) and matrix metalloproteinase 2 (MMP2). Results revealed that tumor cells in neoplastic cerebral organoids expressed higher level of all those GBM invasiveness genes compared to the surrounding normal tissues (FIG. 5f). Interestingly, most invasion-related genes were downregulated in MYC^{OE} neoplastic cerebral organoids compared to GBM groups (FIG. 5e), which correlated with the lower regional infiltration tendency of embryonal neoplasms when compared to astrocytic neoplasms. These observations confirmed the invasiveness of tumor cells from the GBM group of neoplastic cerebral organoids, and suggested the immense potential for using neoplastic cerebral organoids to study the properties of carcinogenic mutations and the behavior of invasive cells at the interface between neoplastic and normal cells.

### Example 7. Screening of EGFR Inhibitors to Reduce Tumor Growth

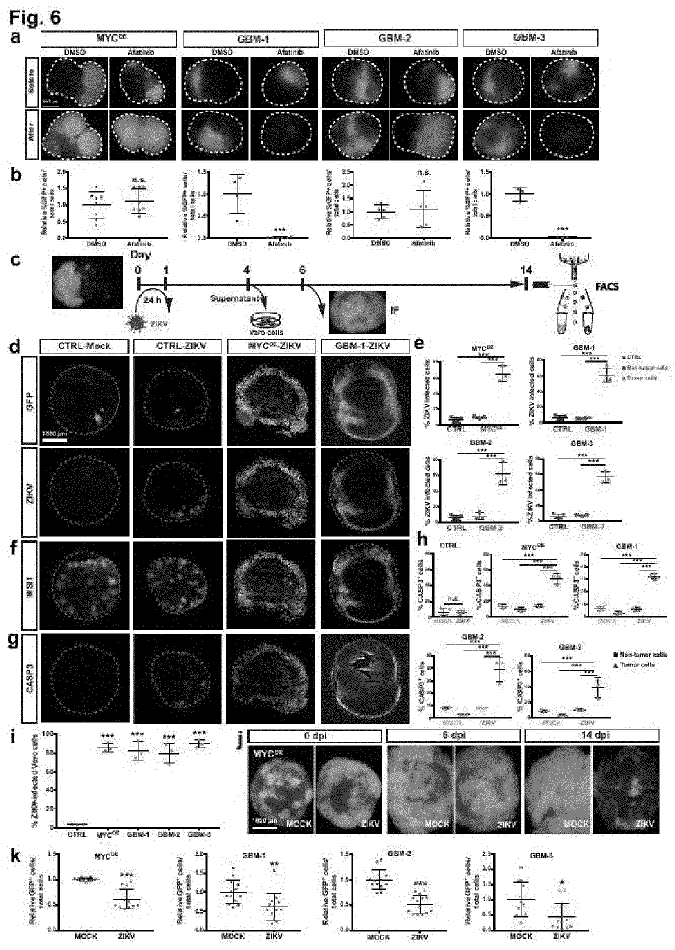

To evaluate the potential use of neoplastic cerebral organoids in preclinical investigation of human brain tumors, we tested the suitability of using the model for targeted drug testing. Since our approach initiated tumorigenesis by introducing defined gene aberrations, the neoplastic cerebral organoids could be potentially used for targeted drug testing. To exam this, we applied one EGFR inhibitor Afatinib, which is currently in a clinical trial for GBM (ClinicalTrials.gov NCT No.: NCT02423525), as a proof of principle. Forty days after treatment, Afatinib significantly reduced the number of tumor cells in GBM-1 and GBM-3 (FIG. 6a,b), but showed no effect on the MYC^{OE} and GBM-2 groups (FIG. 6a,b). This is consistent with the fact that only GBM-1 and GBM-3 organoids are mainly driven by EGFR over-activation. Thus, neoplastic cerebral organoids can be used to test the effect of chemical compounds on tumors originating from specific driver mutations.

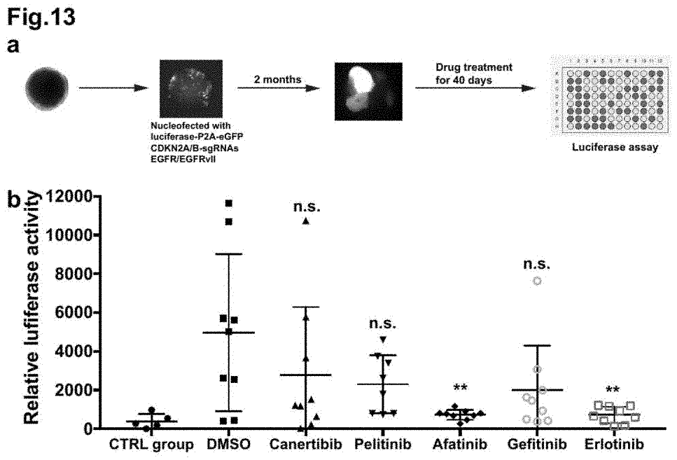

In an effort toward adapting this method for large scale screening, we modified the neoplastic cerebral organoid system to include firefly luciferase for measurement of tumor size (FIG. 13a). Five different EGFR inhibitors, including Afatinib, Erlotinib, and Gefitinib, which are approved for different types of cancers, and the experimental drugs Canertibib and Pelitinib, were applied to organoids from GBM-1 groups, which are mainly driven by EGFR signalling. Forty days after drug treatment, Afatinib and Erlotinib significantly reduced firefly luciferase activity, while the other inhibitors had only non-significant effects (FIG. 13b). Thus, these results suggested that our model could identify the efficacy of different compounds and is suitable for drug screening.

### Example 8. Tumor Tropism and Oncolytic Effect of Zika Virus

Neoplastic cerebral organoids contain both normal and tumor tissues, which make them an ideal model to evaluate tumor tropism and efficacy of oncolytic viral therapy. In this study, we tested the neurotropic ZIKV as the proof of principle. In embryos, ZIKV infects neural precursors resulting in massive apoptosis and severe foetal microcephaly (Qian et al., 2016, Cell, 165, 1238-54; Tang et al., 2016, Cell Stem Cell, 18, 587-90). In adults, the virus causes only mild symptoms and a connection with severe diseases like Guillain-Barre syndrome is controversial (Silva and Souza, 2016, Rev. Soc. Bras. Med. Trop., 49, 267-73). A recent study showed that ZIKV can specifically infect GBM stem cells (Zhu et al., 2017, J. Exp. Med., 214, 2843-57), which shares similarities to NS/PCs (Ward et al., 2007, Annu. Rev. Pathol. Mech. Dis., 2, 175-89).

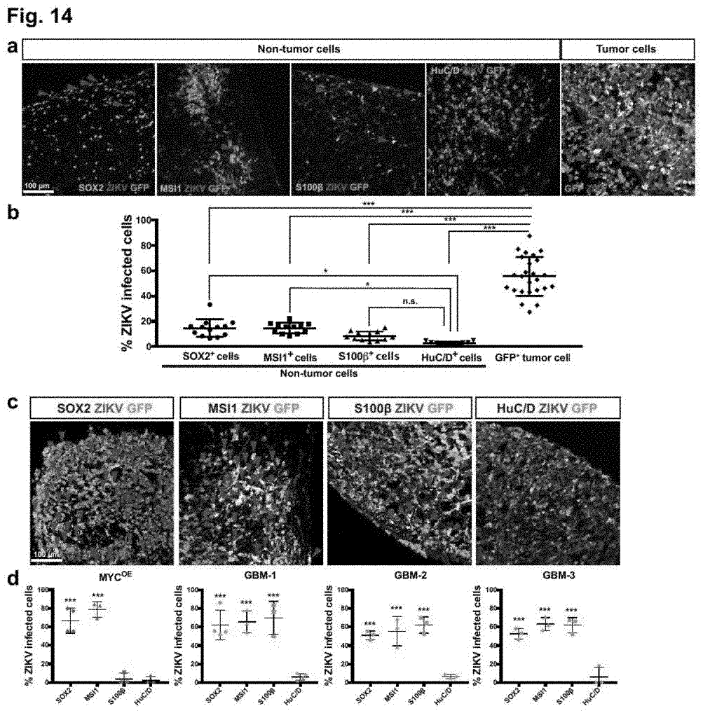

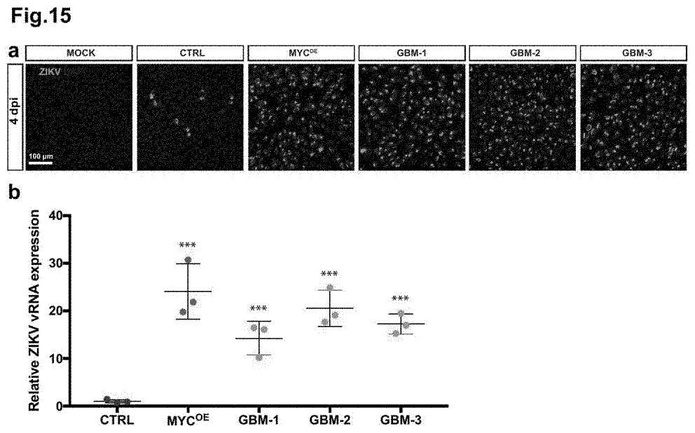

In this study, we used organoids older than 4 months that consist mostly of differentiated neurons and glial cells (Pasca et al., 2015, Nature Methods, 12, 671-8; Renner et al., 2017, EMBO J, 36, 1316-29) (FIG. 6c). Six days post infection (dpi), immunofluorescent analysis from photographs and quantification showed widespread infection of ZIKV in the GFP⁺tumor regions, with little infection in GFP⁻non-tumor regions (FIG. 6d,e). Interestingly, ZIKV⁺cells in the tumor regions expressed the neural precursor markers MUSASHI1 (MSI1) (FIG. 6f), which is also highly expressed in gliomas (Kaneko et al., 2000, Dev. Neurosci., 22, 139-53; Fox et al., 2015, Annu. Rev. Cell Dev. Biol., 31, 249-67). Comparison of ZIKV infection ratio in different subtypes of neural cells in non-tumor regions and GFP⁺tumor cells revealed that ZIKV exhibited higher tropism towards tumor cells than other neural cells, even including NS/PCs in the non-tumor regions (FIG. 14a,b). Further quantification of the cell subtypes infected by ZIKV in tumor regions showed that most ZIKV-infected cells from GBM organoid tissue are SOX2⁺, MSI1⁺NS/PCs, or S100⁺glial cells, but not HuC/D⁺neurons, which is consistent with previous work (Zhu et al., 2017, J.Exp. Med., 214, 2843-57) (FIG. 14c,d). In MYC^{OE} neoplastic cerebral organoids, ZIKV-infected cells are mainly SOX2⁺and MSI1⁺NS/PCs (FIG. 14c,d). In addition, since it has been shown that MSI1 promotes ZIKV replication (Chavali et al., 2017, Science, 357, 83-8), we compared the production of ZIKV particles from CTRL and neoplastic cerebral organoids. This experiment demonstrated that the yield of progeny ZIKV from neoplastic cerebral organoids were significantly higher than CTRL organoids at 4 dpi (FIG. 6i and FIG. 15a,b).

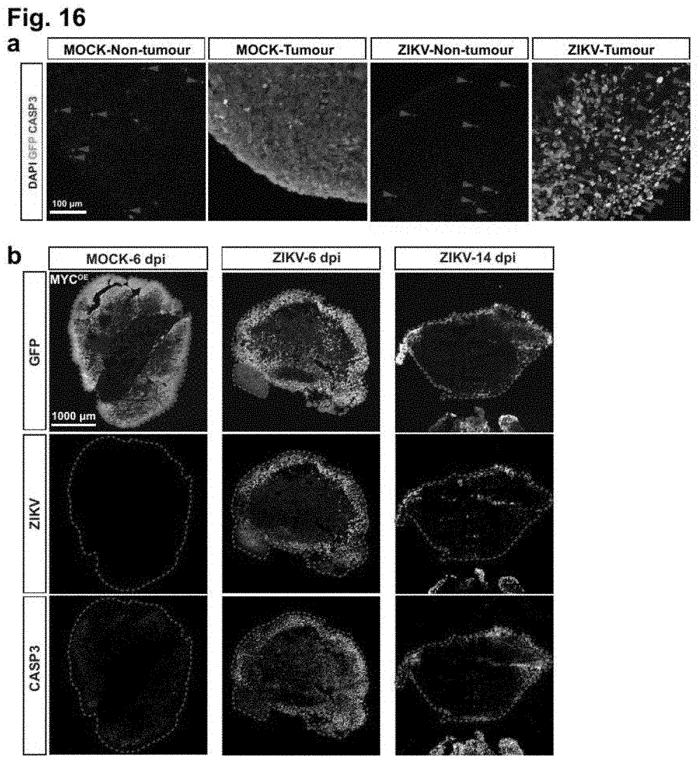

Next, we tested if ZIKV infection could cause tumor cell apoptosis in neoplastic cerebral organoids. We stained for the apoptosis marker activated Caspase3 (CASP3) and found that ZIKV-infected tumor regions in organoids are largely CASP3⁺, while non-tumor regions and CTRL organoids, as well as the MOCK-exposed neoplastic cerebral organoids contained significantly less CASP3⁺cells (FIG. 6g, h and FIG. 16). In the MYC^{OE} group, the oncolytic effect of ZIKV was particularly striking and could even be observed by epifluorescence analysis (FIG. 6j). To further confirm a preferential cytotoxicity of tumor cells over non-tumor cells induced by ZIKV infection, we measured the fraction of GFP⁺cells in neoplastic cerebral organoids at 14 dpi. The proportions of GFP⁺cells in ZIKV-exposed neoplastic cerebral organoids were significantly reduced compared to the proportion in MOCK-exposed neoplastic cerebral organoids (FIG. 6k), indicating that ZIKV exhibits tropism towards tumor cells and significantly reduces the number of tumor cells in both PNET and GBM neoplastic cerebral organoids, with minor damage to normal cells.

### Example 9. Recapitulation and Comparison

By recapitulating genetic aberrations found in human brain cancer patients, we were able to induce tumor-like over proliferation in brain organoids. Neoplastic organoids showed many cancer features, such as cellular identities, cancer pathway specific transcriptome profiles, and capability of in vivo expansion and invasion. We tested three mutant combinations that induce glial-orientated differentiation and abnormal overgrowth, indicating their glial neoplasm-like identities. Furthermore, by overexpressing MYC, we could generate neoplastic organoids that showed histopathological features, cellular identities and transcriptome signatures very similar to those in human CNS-PNET (Sturm et al., 2016, Cell, 164, 1060-70; Ellison et al., 2012, Neuropathology), a tumor for which no successful animal or in vitro model existed so far. It is interesting to note that amplification of MYC alone could initiate CNS-PNET-like neoplasia in cerebral organoids within a very short period, while it requires much longer time in animal models with low incidence (Momota et al., 2008, Oncogene, 27, 4392-401).

Unlike previous GBM culture models (Hubert et al., 2016, Cancer Res, 76: 2465-77), neoplastic cerebral organoids allow the functional analysis of genome aberrations identified in cancer sequencing projects all within the same genetic background. By introducing genome aberrations in organoids started from patient iPS cells, neoplastic organoids can also be used to test the susceptibility of individual patients to different combinations of driver mutations. Unlike glioblastoma cell lines, neoplastic organoids mimic, to a certain degree, the in vivo structural organization. They contain both tumor cells and normal cells within the same culture, so that interactions between transformed and non-transformed cells can be analysed. For drug screening, this particular situation allows for an analysis of anti-tumor effects accompanied by a safety test in the same system. Like all organoid systems, neoplastic organoids are limited by the lack of vasculature so that certain features of GBM such as glomeruloid vascular proliferation and perivascular palisading necrosis are not be observable. Co-culture organoid systems like the one that has been generated for microglia (Muffat et al., 2016, Nat Med, 22, 1358-67) can overcome those limitations.

Our results add ZIKV to the list of oncolytic viruses that might be used to selectively target tumor cells with minimal disruption of non-neoplastic tissues. Viruses from different viral genera and families have been tested in human glioblastoma multiforme and considered for clinical applications against GBM (Russell et al., 2012, Nat Biotechnol., 30, 658-70). ZIKV is a fetal neurotropic virus able to target neural progenitor cells, astrocytes, oligodendrocyte precursors and to a minor extent neurons in the developing fetus (Qian et al., 2016, Cell, 165, 1238-54). Interestingly, our data indicate that the tumor tropism of ZIKV cannot simply be explained by the abundance of immature progenitor cells as a portion of MSI1⁺cells in non-tumor areas or in CTRL organoids were not infected. In adults, the effects of ZIKV infection are mild with only very rare suspected complications (Li et al., 2016, Neuron, 92, 949-58). Thus, a clinical use of ZIKV should be feasible. In any case, our results showcase the power of brain neoplastic organoid models for testing unconventional therapeutic approaches.

1.-15. (canceled)

16. A method of generating an artificial 3D tissue culture of a cancer grown in non-cancerous tissue, comprising the steps of providing an aggregate of pluripotent stem or progenitor cells, culturing and expanding said stem or progenitor cells in a 3D biocompatible matrix, wherein the cells are allowed to differentiate to develop the aggregate into a tissue culture of a desired size; wherein at least a portion of the cells are subjected to carcinogenesis by expressing an oncogene and/or by suppressing a tumor suppressor gene during any of said steps or in the tissue culture, and further comprising the step of allowing said cells with an expressed oncogene or suppressed tumor suppressor gene to develop into cancerous cells.

17. A method of screening a candidate gene or agent for its effects on carcinogenesis, comprising generating an artificial 3D tissue culture, comprising the steps of providing an aggregate of pluripotent stem cells or progenitor, culturing and expanding said stem or progenitor cells in a 3D biocompatible matrix, wherein the cells are allowed to differentiate to develop the aggregate into a tissue culture of a desired size; wherein at least a portion of said cells are subjected to carcinogenesis by expressing or suppressing the candidate gene or by treating the cells with the candidate agent during any of said steps or in the tissue culture, and further comprising the step of culturing said cells in conditions that allow them to develop into cancerous cells.

18. The method of claim 16, wherein the pluripotent stem cells are differentiated into neural cells and/or the tissue is developed into an organoid.

19. The method of claim 16, wherein the 3D biocompatible matrix is a gel, preferably a collagenous gel and/or a hydrogel.

20. The method of claim 16, wherein said aggregate of cells and/or the 3D matrix are cultured in a suspension culture.

21. The method of claim 16, wherein the oncogene, tumor suppressor or candidate gene are selected from CDKN2A, CDKN2B, CDKN2C, NF1, PTEN, p53, ATRX, RB1, CDK4, CDK6, MDM2-B, EGFR, EGFRvIII, PDGFRA, H3F3A, MYC, SMARB1, PTCH1, CTNNB1, MET, RTK, FGFR1, FGFR2, FGFR3, PI3-kinase, PIK3CA, PIK3R1, PIK3C2G, PIK3CB, PIK3C2B, PIK3C2A, PIK3R2, PTEN, BRAF, MDM2, MDM4, MDM1, IDH1, IDH2; preferably from MYC, CDKN2A, CDKN2B, EGFR, EGFRvIII, NF1, PTEN, p53; or combinations thereof such as (i) CDKN2A, CDKN2B, EGFR, and EGFRvIII, (ii) NF1, PTEN and p53, or (iii) EGFRvIII, CDKN2A and PTEN.

22. The method of claim 16, wherein carcinogenesis is after the pluripotent stem cells have been stimulated for tissue-specific differentiation, such as neural differentiation, preferably before expanding said stem cells in a 3D biocompatible matrix, and/or wherein carcinogenesis is a recombinant modification of said genes, preferably by introduction of a transgene for expression of the oncogene or a gene inhibition construct for suppression of the tumor suppressor, especially preferred, wherein said transgene or construct are introduced into cells by nucleofection such as electroporation.

23. The method of claim 16 further comprising the step of identifying cancerous cells in said tissue culture.

24. An artificial 3D tissue culture comprising non-cancerous tissue and cancerous tissue, wherein the cancerous tissue overexpresses an oncogene and/or has a suppressed tumor suppressor, wherein said tissue (i) is obtainable by a method according to claim 16; and/or (ii) comprises a transgene or a construct for suppression of a tumor suppressor at least in cells of the cancerous tissue; and/or (iii) comprises a 3D biocompatible matrix that is a hydrogel.

25. The tissue culture of claim 24, wherein said tissue culture comprises neural tissue and wherein the cancerous tissue is a neural tissue tumor.

26. The tissue culture of claim 24, wherein non-cancerous tissue is at least at the core of the tissue and the cancerous tissue at least at the surface of the tissue.

27. A method of testing or screening a candidate compound or agent or condition for carcinogenesis or for its effect on cancer tissue, comprising contacting cells or a tissue in a method comprising: providing an aggregate of pluripotent stem or progenitor cells, culturing and expanding said stem or progenitor cells in a 3D biocompatible matrix, wherein the cells are allowed to differentiate to develop the aggregate into a tissue culture of a desired size; wherein at least a portion of the cells are subjected to carcinogenesis by expressing an oncogene and/or by suppressing a tumor suppressor gene during any of said steps or in the tissue culture, and further comprising the step of allowing said cells with an expressed oncogene or suppressed tumor suppressor gene to develop into cancerous cells, with the candidate compound or agent or exposing it to the condition, or contacting a tissue of claim 24, with the candidate compound or agent or exposing it to the condition; and maintaining said contacted tissue in culture, and observing any changes in the tissue as compared to said tissue without contacting by said candidate compound or agent or exposure to said condition.

28. The method of claim 27, wherein the candidate agent comprises a virus, preferably a Flavivirus, or wherein the candidate compound comprises a biomolecule, such as a protein or a nucleic acid.

29. The method of claim 27, wherein the condition comprises a difference in culturing environment, preferably lowered or increased nutrients, such as glucose, fat or fatty acids, or altered redox potential or altered temperature.


SEQUENCE LISTING (40 sequences)

SEQ ID NO: 1 - DNA, 40 bp, Artificial Sequence
  primer
gacggcgcgcccgccaccatgctggatttttttcgggtag

SEQ ID NO: 2 - DNA, 30 bp, Artificial Sequence
  primer
gacaccggtttacgcacaagagttccgtag

SEQ ID NO: 3 - DNA, 36 bp, Artificial Sequence
  primer
gacggcgcgcccgccaccatgcgaccctccgggacg

SEQ ID NO: 4 - DNA, 31 bp, Artificial Sequence
  primer
gacaccggttcatgctccaataaattcactg

SEQ ID NO: 5 - DNA, 42 bp, Artificial Sequence
  primer
gacggcgcgcccgccaccatggggacttcccatccggcgttc

SEQ ID NO: 6 - DNA, 33 bp, Artificial Sequence
  primer
gacaccggtttacaggaagctgtcttccaccag

SEQ ID NO: 7 - DNA, 40 bp, Artificial Sequence
  primer
gacggcgcgcccgccaccatggctacctctcgatatgagc

SEQ ID NO: 8 - DNA, 31 bp, Artificial Sequence
  primer
gacaccggttcactccggattaccttcatcc

SEQ ID NO: 9 - DNA, 40 bp, Artificial Sequence
  primer
gacggcgcgcccgccaccatgtgcaataccaacatgtctg

SEQ ID NO: 10 - DNA, 30 bp, Artificial Sequence
  primer
gacaccggtctaggggaaataagttagcac

SEQ ID NO: 11 - DNA, 69 bp, Artificial Sequence
  primer
cattttggcaaagaattccctcgataccgggggcgcgcccgccaccatggctcgtacaaagcagactgc

SEQ ID NO: 12 - DNA, 65 bp, Artificial Sequence
  primer
cgggaatgctagcaatcattggttgatcagctttgttaccggtttaagcacgttctccacgtatg

SEQ ID NO: 13 - DNA, 25 bp, Artificial Sequence
  primer
caccgtcccgggcagcgtcgtgcac

SEQ ID NO: 14 - DNA, 25 bp, Artificial Sequence
  primer
aaacgtgcacgacgctgcccgggac

SEQ ID NO: 15 - DNA, 25 bp, Artificial Sequence
  primer
caccgacggagtcaaccgtttcggg

SEQ ID NO: 16 - DNA, 25 bp, Artificial Sequence
  primer
aaaccccgaaacggttgactccgtc

SEQ ID NO: 17 - DNA, 25 bp, Artificial Sequence
  primer
caccgctcgtcgaagcggctgacca

SEQ ID NO: 18 - DNA, 25 bp, Artificial Sequence
  primer
aaactggtcagccgcttcgacgagc

SEQ ID NO: 19 - DNA, 25 bp, Artificial Sequence
  primer
caccgaacttgtcttcccgtcgtgt

SEQ ID NO: 20 - DNA, 25 bp, Artificial Sequence
  primer
aaacacacgacgggaagacaagttc

SEQ ID NO: 21 - DNA, 25 bp, Artificial Sequence
  primer
caccgtcgacgctaggatctgactg

SEQ ID NO: 22 - DNA, 25 bp, Artificial Sequence
  primer
aaaccagtcagatcctagcgtcgac

SEQ ID NO: 23 - DNA, 25 bp, Artificial Sequence
  primer
caccgcggtggcggccgtttttcgg

SEQ ID NO: 24 - DNA, 25 bp, Artificial Sequence
  primer
aaacccgaaaaacggccgccaccgc

SEQ ID NO: 25 - DNA, 25 bp, Artificial Sequence
  primer
caccgaaattccgagtttcgagcga

SEQ ID NO: 26 - DNA, 25 bp, Artificial Sequence
  primer
aaactcgctcgaaactcggaatttc

SEQ ID NO: 27 - DNA, 24 bp, Artificial Sequence
  primer
caccgagaacctcggaacatacgg

SEQ ID NO: 28 - DNA, 24 bp, Artificial Sequence
  primer
aaacccgtatgttccgaggttctc

SEQ ID NO: 29 - DNA, 25 bp, Artificial Sequence
  primer
caccgcagatagtcccggtccggcg

SEQ ID NO: 30 - DNA, 25 bp, Artificial Sequence
  primer
aaaccgccggaccgggactatctgc

SEQ ID NO: 31 - DNA, 25 bp, Artificial Sequence
  primer
caccgaaacagctcgttgtaccgct

SEQ ID NO: 32 - DNA, 25 bp, Artificial Sequence
  primer
aaacagcggtacaacgagctgtttc

SEQ ID NO: 33 - DNA, 20 bp, Artificial Sequence
  primer
tcggattctctgctctcctc

SEQ ID NO: 34 - DNA, 20 bp, Artificial Sequence
  primer
cctgcctcttttccacagaa

SEQ ID NO: 35 - DNA, 18 bp, Artificial Sequence
  primer
cgggctctggaggaaaag

SEQ ID NO: 36 - DNA, 20 bp, Artificial Sequence
  primer
gcccttcgcacttcttacac

SEQ ID NO: 37 - DNA, 20 bp, Artificial Sequence
  primer
gggcaccactccactgtatc

SEQ ID NO: 38 - DNA, 21 bp, Artificial Sequence
  primer
cgaagtgcaatggtctttagg

SEQ ID NO: 39 - DNA, 23 bp, Artificial Sequence
  primer
ttggtcatgatactgctgattgc

SEQ ID NO: 40 - DNA, 22 bp, Artificial Sequence
  primer
ccttccacaaagtccctattgc